(19) United States
(12) Patent Application Publication
(10) Pub. No.: US 2020/0383943 A1
(43) Pub. Date: Dec. 10, 2020

(54) METHODS OF CANCER TREATMENT VIA REGULATED FERROPTOSIS

(71) Applicants: Memorial Sloan Kettering Cancer Center, New York, NY (US); Comell University, Ithaca, NY (US)

(72) Inventors: Michelle S. Bradbury, New York, NY (US); Michael Overholtzer, Glen Ridge, NJ (US); Howard Scher, Tenafly, NJ (US); Ulrich Wiesner, Ithaca, NY (US); Brian Madajewski, New York, NY (US)

(21) Appl. No.: 16/769,501

(22) PCT Filed: Dec. 4, 2018
(86) PCT No.: PCT/US18/63751

§ 371 Date: Jun. 3, 2020

Related U.S. Application Data
(60) Provisional application No. 62/771,468, filed on Nov. 26, 2018.
(60) Provisional application No. 62/594,499, filed on Dec. 4, 2017.

(51) Int. Cl. A61K 31/198; A61P 35/00; A61K 47/69

(52) U.S. Cl. CPC A61K 31/198; A61K 45/06; A61K 47/6929; A61P 35/00

(57) ABSTRACT

The present disclosure describes methods of treatment (e.g., combination treatment) by ferroptotic induction, as well as compositions and dosing regimens that are part of such methods. Surprisingly, it is presently found that delaying administration of a ferroptosis-inducing agent until after starting hormone therapy results in enhanced ferroptotic induction in a subject. Thus, in certain embodiments, combination therapies are presented herein that include multiple administration steps whereby a ferroptosis-inducing agent is administered some time after hormone therapy has begun.

### CROSS-REFERENCE TO RELATED APPLICATION

This application claims the benefit of U.S. Application Ser. No. 62/594,499 filed on Dec. 4, 2017 and U.S. Application Ser. No. 62/771,468 filed on Nov. 26, 2018, the disclosures of which are hereby incorporated by reference in their entireties.

### GOVERNMENT SUPPORT

This invention was made with government support under grant numbers CA199081 and GM111350 awarded by National Institutes of Health. The government has certain rights in the invention.

### FIELD OF THE INVENTION

This invention relates generally to methods and compositions for the treatment of cancer in subjects.

### SUMMARY

It is presently found that ferroptosis occurs in a regulated manner and likely involves the formation of a plasma membrane pore. It is also found that ferroptosis involves the spreading of cell death between adjacent cells in wave-like or synchronous patterns. Surprisingly, it is presently found that delaying administration of a ferroptosis-inducing agent until after starting hormone therapy in a subject with cancer, e.g., castrate-resistant prostate cancer, results in enhanced ferroptotic induction in the subject. Thus, in certain embodiments, combination therapies are presented herein which include multiple administration steps whereby a ferroptosis-inducing agent is administered some time after hormone therapy has begun. Furthermore, by performing high content, image-based screening of ferroptotic cell death in response to cell treatment, genetic and/or chemical modulators of ferroptotic parameters (e.g., describing the spreading of cell death) can be identified for improved ferroptotic induction. In certain embodiments, the image-based screening makes use of super-resolution optical imaging and/or nanosensor technologies. Dosing regimens in combination therapies may also be optimized via such image-based screening.

The present disclosure describes methods of treatment (e.g., combination treatment) by ferroptotic induction, as well as compositions and dosing regimens that are part of such methods. For example, a subject undergoing hormone therapy for treatment of disease, such as prostate cancer (e.g., a subject who has developed castration resistance, e.g., a subject with castrate-resistant prostate cancer, CRPC) exhibits increased expression of PSMA. Surprisingly, it is presently found that delaying administration of a ferroptotic inducing agent, such as PSMA-targeting C′ dots, after starting hormone therapy results in enhanced ferroptotic induction in a subject.

The present disclosure describes that, while PSMAi-PEG-C′ dots induced a modest ferroptotic induction, their combination with enzalutamide in vitro was synergistic in PSMA-expressing LNCaP lines. As a result, in vivo significant tumor volume reductions were sustained (e.g., tumor shrinkage was maintained) with both MC1-R-targeting and PSMA-targeting C′ dots (see, e.g., the results in FIG. 6, which demonstrates that the administration of functionalized (PSMAi, αMSH) ferroptotic agents such as C′ dots at Day 0, 3, and 6 result in a significant inhibition of LNCAP tumor growth when compared to vehicle control at Day 9; ** p=0.0021; ***p=0.0002). Thus, data supports that the present methods meet a key clinical need for maintaining tumor regression and durability.

Thus, in certain embodiments, methods of combination treatment of a subject are enabled and described herein, which take advantage of this presently discovered synergistic effect.

In one aspect, the invention is directed to a method of combination treatment of a subject (e.g., a subject having been diagnosed with cancer, e.g., prostate cancer), the method comprising: a first step of administering an initial dose of a first agent to the subject; and a second step of administering an initial dose of a second agent to the subject to induce ferroptosis of cancer cells (e.g., prostate cancer, e.g., castrate-resistant prostate cancer CRPC), wherein the step of administering the initial dose of the second agent occurs at a discrete period of time after the step of administering the initial dose of the second agent (e.g., a known period of time, e.g., greater than 12 hours, e.g., greater than 1 day, e.g., greater than 2 days, e.g., about 5 days, e.g., greater than 5 days, e.g., greater than 7 days, e.g., greater than 10 days, e.g., about 15 days).

In certain embodiments, the first agent comprises an androgen inhibitor or other agent administered as part of hormone therapy.

In certain embodiments, the method comprises administering the first agent to the subject on a daily basis.

In certain embodiments, the first agent comprises an androgen inhibitor and wherein the androgen inhibitor causes increased expression of PSMA by prostate cancer cells (e.g., increased expression by prostate cancer cells than by normal prostate cells). In certain embodiments, the second agent comprises a ferroptosis-inducing nanoparticle with a PSMA-targeting ligand.

In certain embodiments, the increased expression of PSMA by the prostate cancer cells results in enhanced ferroptotic induction by the second agent, e.g., due to improved targeting of the ferroptosis-inducing nanoparticle to the prostate cancer cells.

In certain embodiments, administering the second agent induces ferroptotic rupture of the cancer cells.

In certain embodiments, the method comprises inducing a pore-forming activity and/or a spreading activity, e.g., thereby resulting in a spreading of cell death in wave-like and/or synchronous patterns, e.g., thereby resulting in a controlled spreading of cell death of cancer cells. In certain embodiments, the method comprises maintaining the cancer cells in a nutrient-deprived environment, or, alternatively, not maintaining the cancer cells in a nutrient-deprived environment. In certain embodiments, the method comprises administering one or more regulators of ferroptosis. In certain embodiments, the one or more regulators of ferroptosis comprise one or more activators of ferroptosis and/or one or more inhibitors of ferroptosis. In certain embodiments, the one or more activators of ferroptosis comprises one or more members selected from the group consisting of (i) RSL3 and/or other compounds which inhibit GPX4, (ii) erastin (e.g., and/or another compound which inhibits amino acid transporters system xc−), and (iii) buthionine sulfoximine (BSO) which inhibits gamma-glutamylcysteine synthetase and production of glutathione. In certain embodiments, the one or more inhibitors of ferroptosis comprises a member selected from the group consisting of liproxstatin-1, ferrostatin-1, and/or other compounds which scavenge lipid peroxides. In certain embodiments, the one or more regulators of ferroptosis control spreading of ferroptotic cell death in a tissue of the subject, e.g., thereby activating and/or accelerating and/or enhancing cancer cell death (and/or the spreading of cancer cell death) by ferroptosis and/or thereby inhibiting and/or preventing ferroptotic cell death of non-cancerous cells. In certain embodiments, the one or more regulators of ferroptosis is different from the second agent that induces ferroptosis, or, alternatively, wherein the one or more regulators of ferroptosis includes the second agent that induces ferroptosis.

In certain embodiments, the second agent comprises a nanoparticle. In certain embodiments, the second agent comprises an inhibitor-functionalized ultrasmall nanoparticle as described in International Patent Application No. PCT/US17/63641, “Inhibitor-Functionalized Ultrasmall Nanoparticles and Methods Thereof,” filed Nov. 29, 2017, published as WO/2018/102372, the text of which is incorporated herein by reference in its entirety. In certain embodiments, the nanoparticle has from 1 to 100 targeting ligands (e.g., from 1 to 80, e.g., from 1 to 60, e.g., from 1 to 40, e.g., from 1 to 30, e.g., from 1 to 20 targeting ligands) attached thereto. In certain embodiments, the targeting ligands comprise a member selected from the group comprising of PSMAi and alpha-MSH. In certain embodiments, the nanoparticle comprises a radiolabel. In certain embodiments, the nanoparticle has an average diameter no greater than about 50 nm (e.g., no greater than about 40 nm, e.g., no greater than about 30 nm, e.g., no greater than about 25 nm, e.g., no greater than about 20 nm, e.g., no greater than about 15 nm, e.g., no greater than about 10 nm, e.g., no greater than about 8 nm).

In certain embodiments, the second agent does not comprise a nanoparticle. In certain embodiments, the second agent comprises (e.g., is) a small molecule. In certain embodiments, the second agent comprises (e.g., is) erastin. In certain embodiments, the second agent comprises iron (e.g., excess iron). In certain embodiments, the second agent comprises (e.g., is) an inhibitor of enzyme GPX4. In certain embodiments, the inhibitor of enzyme GPX4 comprises a member selected from the group consisting of RLS3 and ML162. In certain embodiments, the second agent comprises (e.g., is) buthionine sulfoximine (BSO) and/or another compound which induces ferroptosis by inhibiting production of glutathione.

In certain embodiments, the second agent comprises a nanoparticle and a species associated with (e.g., bound to) the nanoparticle.

In certain embodiments, the method comprises administering one or more members of the group consisting of a molecular agent, a free drug, a nanoparticle-bound agent, and a nanoparticle-bound drug.

In certain embodiments, the method comprises administering the second agent to the subject for accumulation at sufficiently high concentration in cancer cells to induce ferroptosis (e.g., ferroptotic cell death involving iron-dependent necrosis or reactive oxygen species-dependent necrosis).

In certain embodiments, the cancer cells is selected from the group consisting of renal, prostate, melanoma, pancreatic, lung, fibrosarcoma, breast, brain, ovarian, and colon cancer cells.

In certain embodiments, the combination treatment further comprises administering to the subject (i) one or more standard-of-care ICB antibodies (e.g., intact antibodies, such as anti-PD-1 or PD-L1, and/or their fragments) and/or one or more small molecule inhibitors; and/or (ii) one or more standard-of-care anti-androgen receptor therapeutics and/or a hypoxia-activated prodrug. In certain embodiments, (i) and/or (ii) is attached to a nanoparticle or other carrier, e.g., for maximizing biological properties, e.g., targeted uptake.

In certain embodiments, the hormone therapy comprises a member selected from the group consisting of (i) treatments to lower androgen levels (e.g., Orchiectomy (surgical castration), luteinizing hormone-releasing hormone (LHRH) agonists, LHRH antagonists (e.g., Degarelix (Firmagon), CYP17 inhibitors, and/or Abiraterone (Zytiga)), (ii) treatments to stop androgens from working (e.g., antiandrogens such as Enzalutamide, Apalutamide, Darolutamide, cyroterone, Androcur, Nilutamide, Bicalutamide), and (iii) other androgen-suppressing drugs (e.g., estrogens, ketoconazole).

In certain embodiments, the method further comprises administering systemic therapy (e.g., chemotherapy, hormonal therapy, targeted drugs, and immunotherapy), e.g., wherein the method enhances antitumor effects of a standard therapy.

In certain embodiments, the method comprises monitoring (e.g., continuously, e.g., in real-time, e.g., during surgery), via a detector, responses of the subject to treatment via one or more imaging modalities (e.g., positron emission tomography (PET), single-photon emission computed tomography (SPECT), computed tomography (CT), and/or fluorescence imaging). In certain embodiments, the method comprises placing one or more clips in the body of a patient (e.g., during a surgical procedure) to guide later-administered therapy (e.g., radiotherapy).

In certain embodiments, the method comprises monitoring (e.g., continuously, e.g., in real-time, e.g., during surgery), via a detector, responses of the subject to treatment by detecting one or more environmental conditions and/or analytes selected from the group consisting of reactive oxygen species (ROS), pH, pH perturbation, iron level, calcium, glutathione, leucine, glutamine, arginine, and other amino acid via a readout on the detector (e.g., a 2D or 3D map of the detected environmental condition and/or analyte level).

In certain embodiments, the method comprises performing super-resolution microscopy to identify administered nanoparticles in tissue of the subject on a sub-cellular level (e.g., an organelle or sub-organelle level, e.g. at a resolution of). In certain embodiments, the method comprises (i) assessing nanoparticle delivery and/or trafficking and/or (ii) nanosensor imaging of cancer metabolism and/or therapeutic response and/or progression, e.g., thereby informing therapy adjustment.

In certain embodiments, the method comprises optimizing the dose or frequency of the administration of the first agent to maximize expression of PSMA.

In another aspect, the invention is directed to a method of combination treatment of a subject (e.g., a subject having been diagnosed with cancer, e.g., prostate cancer), the method comprising: a first step of administering an initial dose of a first agent to the subject; and a second step of administering an initial dose of a second agent to the subject to induce ferroptosis of cancer cells (e.g., prostate cancer), wherein the step of administering the initial dose of the second agent occurs at a discrete period of time after the step of administering the initial dose of the second agent (e.g., a known period of time, e.g., greater than 12 hours, e.g., greater than 1 day, e.g., greater than 2 days, e.g., about 5 days, e.g., greater than 5 days, e.g., greater than 7 days, e.g., greater than 10 days, e.g., about 15 days) (e.g., wherein the first agent comprises an androgen inhibitor and wherein the androgen inhibitor causes increased expression of PSMA by prostate cancer cells (e.g., increased expression by prostate cancer cells than by normal prostate cells), e.g., wherein the second agent comprises a ferroptosis-inducing nanoparticle with a PSMA-targeting ligand, e.g., wherein the increased expression of PSMA by the prostate cancer cells results in enhanced ferroptotic induction by the second agent, e.g., due to improved targeting of the ferroptosis-inducing nanoparticle to the prostate cancer cells), wherein the subject has also received an androgen inhibitor, e.g., via one or more separate doses.

In certain embodiments, the first agent comprises an androgen inhibitor or other agent administered as part of hormone therapy.

In certain embodiments, the method comprises administering the first agent to the subject on a daily basis. In certain embodiments, the first agent comprises an androgen inhibitor and wherein the androgen inhibitor causes increased expression of PSMA by prostate cancer cells (e.g., increased expression by prostate cancer cells than by normal prostate cells). In certain embodiments, the second agent comprises a ferroptosis-inducing nanoparticle with a PSMA-targeting ligand. In certain embodiments, the increased expression of PSMA by the prostate cancer cells results in enhanced ferroptotic induction by the second agent, e.g., due to improved targeting of the ferroptosis-inducing nanoparticle to the prostate cancer cells.

In certain embodiments, the combination treatment comprises radiotherapy.

In another aspect, the invention is directed to a kit comprising: a first agent in a unit dosage effective to treat prostate cancer in a subject receiving therapy with the first agent; and a second agent. In certain embodiments, the first agent comprises an androgen inhibitor or other agent administered as part of hormone therapy. In certain embodiments, the androgen inhibitor maximizes PSMA expression. In certain embodiments, the second agent comprises a ferroptosis-inducing nanoparticle with a PSMA-targeting ligand.

In another aspect, the invention is directed to a treatment comprising a therapeutically effective amount of a first agent (e.g., comprising an androgen inhibitor or other agent administered as part of hormone therapy) for use in combination with a second agent (e.g., wherein the second agent comprises a ferroptosis-inducing nanoparticle with a PSMA-targeting ligand) for use in a method of treating cancer (e.g., prostate cancer) or preventing cancer occurrence or recurrence in a subject.

In another aspect, the invention is directed to a method of treating cancer in a subject (e.g., a subject having been diagnosed with cancer), the method comprising: administering a composition to the subject to induce ferroptosis of cancer cells (e.g., to induce ferroptotic rupture of the cancer cells, e.g., to induce a pore-forming activity and/or a spreading activity, e.g., thereby resulting in a spreading of cell death in wave-like and/or synchronous patterns, e.g., thereby resulting in a controlled spreading of cell death of cancer cells) (e.g., wherein the method comprises maintaining the cancer cells in a nutrient-deprived environment, or, alternatively, not maintaining the cancer cells in a nutrient-deprived environment).

In certain embodiments, the composition comprises a nanoparticle.

In certain embodiments, the composition does not comprise a nanoparticle (e.g., wherein the composition comprises (e.g., is) a small molecule, e.g., wherein the composition comprises (e.g., is) erastin, e.g., wherein the composition comprises iron (e.g., excess iron), e.g., wherein the composition comprises (e.g., is) RSL3 (e.g., an inhibitor of the enzyme GPX4), e.g. wherein the composition comprises (e.g., is) buthionine sulfoximine (BSO) and/or another compound which induces ferroptosis by inhibiting production of glutathione)).

Elements of embodiments involving one aspect of the invention (e.g., methods) can be applied in embodiments involving other aspects of the invention (e.g., compositions), and vice versa.

### Definitions

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In certain embodiments, administration is oral. Additionally or alternatively, in certain embodiments, administration is parenteral. In certain embodiments, administration is intravenous.

“Agent”: The term “agent”, as used herein, may refer to a compound, molecule, or entity of any chemical and/or biological class including, for example, a small molecule, polypeptide, nucleic acid, saccharide, lipid, metal, or a combination or complex thereof. In certain embodiments, the term “agent” may refer to a compound, molecule, or entity that comprises a polymer. In certain embodiments, the term may refer to a compound or entity that comprises one or more polymeric moieties. In certain embodiments, the term may refer to a compound, molecule, or entity that lacks or is substantially free of any polymer or polymeric moiety. In some embodiments, the term may refer to a nanoparticle.

“Antibody”: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload[e.g., a detectable moiety, a therapeutic moiety, a catalytic moiety, etc], or other pendant group [e.g., poly-ethylene glycol, and the like]

“Antibody agent”: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc, as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, and the like]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

“Biocompatible”: The term “biocompatible”, as used herein is intended to describe materials that do not elicit a substantial detrimental response in vivo. In certain embodiments, the materials are “biocompatible” if they are not toxic to cells. In certain embodiments, materials are “biocompatible” if their addition to cells in vitro results in less than or equal to 20% cell death, and/or their administration in vivo does not induce inflammation or other such adverse effects. In certain embodiments, materials are biodegradable.

“Biodegradable”: As used herein, “biodegradable” materials are those that, when introduced into cells, are broken down by cellular machinery (e.g., enzymatic degradation) or by hydrolysis into components that cells can either reuse or dispose of without significant toxic effects on the cells. In certain embodiments, components generated by breakdown of a biodegradable material do not induce inflammation and/or other adverse effects in vivo. In certain embodiments, biodegradable materials are enzymatically broken down. Alternatively or additionally, in certain embodiments, biodegradable materials are broken down by hydrolysis. In certain embodiments, biodegradable polymeric materials break down into their component polymers. In certain embodiments, breakdown of biodegradable materials (including, for example, biodegradable polymeric materials) includes hydrolysis of ester bonds. In certain embodiments, breakdown of materials (including, for example, biodegradable polymeric materials) includes cleavage of urethane linkages.

“Cancer”: As used herein, the term “cancer” refers to a malignant neoplasm or tumor (Stedman's Medical Dictionary, 25th ed.; Hensly ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B cell ALL, T cell ALL), acute myelocytic leukemia (AML) (e.g., B cell AML, T cell AML), chronic myelocytic leukemia (CML) (e.g., B cell CIVIL, T cell CML), and chronic lymphocytic leukemia (CLL) (e.g., B cell CLL, T cell CLL)); lymphoma such as Hodgkin lymphoma (HL) (e.g., B cell HL, T cell HL) and non Hodgkin lymphoma (NHL) (e.g., B cell NHL such as diffuse large cell lymphoma (DLCL) (e.g., diffuse large B cell lymphoma), follicular lymphoma, chronic lymphocytic leukemia/small lymphocytic lymphoma (CLL/SLL), mantle cell lymphoma (MCL), marginal zone B cell lymphomas (e.g., mucosa associated lymphoid tissue (MALT) lymphomas, nodal marginal zone B cell lymphoma, splenic marginal zone B cell lymphoma), primary mediastinal B cell lymphoma, Burkitt lymphoma, lymphoplasmacytic lymphoma (e.g., Waldenstrom's macroglobulinemia), hairy cell leukemia (HCL), immunoblastic large cell lymphoma, precursor B lymphoblastic lymphoma and primary central nervous system (CNS) lymphoma; and T cell NHL such as precursor T lymphoblastic lymphoma/leukemia, peripheral T cell lymphoma (PTCL) (e.g., cutaneous T cell lymphoma (CTCL) (e.g., mycosis fungoides, Sezary syndrome), angioimmunoblastic T cell lymphoma, extranodal natural killer T cell lymphoma, enteropathy type T cell lymphoma, subcutaneous panniculitis like T cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CIVIL), chronic neutrophilic leukemia (CNL), hypereosinophilic syndrome (HES); neuroblastoma; neurofibroma (e.g., neurofibromatosis (NF) type 1 or type 2, schwannomatosis); neuroendocrine cancer (e.g., gastroenteropancreatic neuroendocrine tumor (GEP NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

“Carrier”: As used herein, “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

“Chemotherapeutic Agent”: As used herein, the term “chemotherapeutic agent” or “oncolytic therapeutic agent” (e.g., anti-cancer drug, e.g., anti-cancer therapy, e.g., immune cell therapy) has its art-understood meaning referring to one or more pro-apoptotic, cytostatic and/or cytotoxic agents, and/or hormonal agents, for example, specifically including agents utilized and/or recommended for use in treating one or more diseases, disorders or conditions associated with undesirable cell proliferation. In many embodiments, chemotherapeutic agents and/or oncolytic therapeutic agents are useful in the treatment of cancer. In some embodiments, a chemotherapeutic agent and/or oncolytic therapeutic agents may be or comprise one or more hormonal agents (e.g., androgen inhibitors), one or more alkylating agents, one or more anthracyclines, one or more cytoskeletal disruptors (e.g., microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some particular embodiments, a chemotherapeutic agent may be or comprise one or more of Actinomycin, all-trans retinoic acid, an Auiristatin, Azacitidine, Azathioprine, Bleomycin, Bortezomib, Carboplatin, Capecitabine, Cisplatin, Chlorambucil, Cyclophosphamide, curcumin, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Maytansine and/or analogs thereof (e.g., DM1) Mechlorethamine, Mercaptopurine, Methotrexate, Mitoxantrone, a Maytansinoid, Oxaliplatin, Paclitaxel, Pemetrexed, Teniposide, Tioguanine, Topotecan, Valrubicin, Vinblastine, Vincristine, Vindesine, Vinorelbine, and combinations thereof. In some embodiments, a chemotherapeutic agent may be utilized in the context of an antibody-drug conjugate. In some embodiments, a chemotherapeutic agent is one found in an antibody-drug conjugate selected from the group consisting of: hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and lorvotuzumab mertansine. In some embodiments, a chemotherapeutic agent may be or comprise one or more of farnesyl-thiosalicylic acid (FTS), 4-(4-Chloro-2-methylphenoxy)-N-hydroxybutanamide (CMH), estradiol (E2), tetramethoxystilbene (TMS), δ-tocatrienol, salinomycin, or curcumin. In certain embodiments, chemotherapeutic agents and/or oncolytic therapeutic agents for anti-cancer treatment comprise (e.g., are) biological agents such as tumor-infiltrating lymphocytes, CAR T-cells, antibodies, antigens, therapeutic vaccines (e.g., made from a patient's own tumor cells or other substances such as antigens that are produced by certain tumors), immune-modulating agents (e.g., cytokines, e.g., immunomodulatory drugs or biological response modifiers), checkpoint inhibitors) or other immunologic agents. In certain embodiments, immunologic agents include immunoglobins, immunostimulants (e.g., bacterial vaccines, colony stimulating factors, interferons, interleukins, therapeutic vaccines, vaccine combinations, viral vaccines) and/or immunosuppressive agents (e.g., calcineurin inhibitors, interleukin inhibitors, TNF alpha inhibitors). In certain embodiments, hormonal agents include agents for anti-androgen therapy (e.g., Ketoconazole, ABiraterone, TAK-700, TOK-001, Bicalutamide, Nilutamide, Flutamide, Enzalutamide, ARN-509).

“Combination Treatment” or “Combination Therapy”: As used herein, the term “combination treatment”, which is interchangeable with the term “combination therapy”, refers to those situations in which a subject is simultaneously exposed to two or more therapeutic regimens (e.g., involving two or more therapeutic agents). In some embodiments, two or more agents may be administered simultaneously; in some embodiments, such agents may be administered sequentially; in some embodiments, such agents are administered in overlapping dosing regimens.

“Peptide” or “Polypeptide”: The term “peptide” or “polypeptide” refers to a string of at least two (e.g., at least three) amino acids linked together by peptide bonds. In certain embodiments, a polypeptide comprises naturally-occurring amino acids; alternatively or additionally, in certain embodiments, a polypeptide comprises one or more non-natural amino acids (i.e., compounds that do not occur in nature but that can be incorporated into a polypeptide chain; see, for example, http://www.cco.caltech.edu/\tilde{ }dadgrp/Unnatstruct.gif, which displays structures of non-natural amino acids that have been successfully incorporated into functional ion channels) and/or amino acid analogs as are known in the art may alternatively be employed). In certain embodiments, one or more of the amino acids in a protein may be modified, for example, by the addition of a chemical entity such as a carbohydrate group, a phosphate group, a farnesyl group, an isofarnesyl group, a fatty acid group, a linker for conjugation, functionalization, or other modification.

“Radiolabel”: As used herein, “radiolabel” refers to a moiety comprising a radioactive isotope of at least one element. Exemplary suitable radiolabels include but are not limited to those described herein. In certain embodiments, a radiolabel is one used in positron emission tomography (PET). In certain embodiments, a radiolabel is one used in single-photon emission computed tomography (SPECT). In certain embodiments, radioisotopes comprise ^{99m}Tc, ¹¹¹In, ⁶⁴Cn, ⁶⁷Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Ab, ¹⁹⁹Ab, ¹⁶⁹Yb, ¹⁷⁵Yb, ¹⁶⁵Dy, ¹⁶⁶Dy, ⁶⁷Cu, ¹⁰⁵Rh, ¹¹¹Ag, ⁸⁹Zr, ²²⁵Ac, and ¹⁹²Ir.

“Subject”: As used herein, the term “subject” includes humans and mammals (e.g., mice, rats, pigs, cats, dogs, and horses). In many embodiments, subjects are mammals, particularly primates, especially humans. In certain embodiments, subjects are livestock such as cattle, sheep, goats, cows, swine, and the like; poultry such as chickens, ducks, geese, turkeys, and the like; and domesticated animals particularly pets such as dogs and cats. In certain embodiments (e.g., particularly in research contexts) subject mammals will be, for example, rodents (e.g., mice, rats, hamsters), rabbits, primates, or swine such as inbred pigs and the like.

“Substantially”: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

“Therapeutic agent”: As used herein, the phrase “therapeutic agent” refers to any agent that has a therapeutic effect and/or elicits a desired biological and/or pharmacological effect, when administered to a subject.

“Treatment”: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In certain embodiments, treatment may be of a subject who has been diagnosed as suffering from the relevant disease, disorder, and/or condition. In certain embodiments, treatment may be of a subject known to have one or more susceptibility factors that are statistically correlated with increased risk of development of the relevant disease, disorder, and/or condition.

Drawings are presented herein for illustration purposes, not for limitation.

### BRIEF DESCRIPTION OF DRAWINGS

The foregoing and other objects, aspects, features, and advantages of the present disclosure will become more apparent and better understood by referring to the following description taken in conduction with the accompanying drawings, in which:

FIGS. 1A-1G depict images and data showing that ferroptosis occurs in a regulated manner consistent with the presence of a plasma membrane pore.

FIG. 1A shows images that cells swell prior to rupture during ferroptosis. Images show 15 and 25 hours post treatment with FAC and BSO. Note cPLA2-mKate protein translocates to the nuclear membrane prior to cell rupture, which indicates cell swelling (bottom images).

FIG. 1B shows a table including osmoprotectants described herein and their known sizes.

FIG. 1C depicts a graph that shows % dead HeLa cells (Sytox green dye-positive) after 48 hours of treatment with FAC and BSO (40 μM). The presence of 30 mM osmoprotectants does not inhibit cell death.

FIGS. 1D-1E shows graphs depicting that the addition of PEG1450 or PEG3350 (30 mM) inhibits cell swelling (FIG. 1D) and cell lysis (FIG. 1E) after 48 hours of treatment with FAC and BSO (400 μM). The effect on cell lysis is shown by reduced release of lactate dehydrogenase (LDH), a measure of necrosis.

FIGS. 1F-1G show graphs depicting that PEG1450 and PEG3350 (30 mM) inhibit cell rupture in response to other inducers of ferroptosis including RSL3 and erastin (HT1080 cells) (FIG. 1F), and ML162 (HAP1 cells) (FIG. 1G).

FIGS. 2A-2D show plots depicting a quantitative approach to measure cell death spreading.

FIG. 2A shows an image depicting nuclei of dead cells (Sytox green dye) in FAC and BSO-treated culture. Nuclei are circled and color-coded to represent the relative timing of cell death, determined by time-lapse microscopy. The relative positioning of cell deaths occurring in successive frames of a time-lapse movie are calculated by measuring the distance between newly occurring deaths and their nearest neighbor of any previous deaths, for example death 2 to death 1, or distance a. The mean distance of all deaths in a movie is calculated as distances (a+b+c+ . . . +n)/n.

FIG. 2B shows an image depicting that, once all deaths in a field of view are calculated as per (FIG. 2A), the order of deaths is computationally shuffled (inset) over 1000 random trials, to generate a library of random possible distances in a given field of view.

FIG. 2C shows (top) a data set for experimentally-induced apoptosis (by treatment with cyclohexamide). The observed mean distance (red) falls within the random trials. FIG. 2C also shows (bottom) a data for ferroptosis induced by treatment with FAC and BSO of MCF10A cells show experimental value well below the random trials, indicating wave-like spreading.

FIG. 2D shows ferroptosis values for FAC and BSO-treated B16F10 (top) and MCF-7 cells (bottom) show significantly non-random patterns (p=0.0026 and p=0.0015).

FIGS. 3A-3C shows images and data depicting that ferroptosis spreading requires lipid peroxidation and occurs independently of cell rupture.

FIG. 3A depicts images that show cell colony prior to (left) and 12 hours after (right) treatment with liproxstatin-1. Note cell death involving wave-like spreading that was induced by FAC and BSO, and started prior to liproxstatin-1 addition, was inhibited, as cells that were alive (bottom right half of colony) stayed alive for 12 hours. The spreading of cell rounding was also blocked by liproxstatin-1.

FIG. 3B shows images and a graph depicting that treatment with PEG1450 does not inhibit wave-like spreading of morphological changes (left image, arrow) in cells treated with FAC and BSO, even though cell rupture is inhibited. Right images show computational quantifications of the spreading of cell rounding (top), which remains significant in the presence of PEG1450 (***P=0.004) (see histogram, bottom). Cells are pseudocolored in top image to represent the relative timing of cell rounding.

FIG. 3C shows images and a graph depicting that cells treated with PEG3350 to block cell rupture still exhibit spreading of cell rounding and also exhibit a wave of increased intracellular calcium, imaged by expression of the GCaMP fluorescent reporter (green). Bottom histogram shows quantification of the non-random pattern of spreading of GCaMP fluorescence (red arrow). Relative times are shown as minutes (min).

FIG. 4 shows images depicting a cell system for screening of ferroptosis modulators. Top images: cells expressing GFP and treated with Sytox orange dye (red) exhibit loss of GFP fluorescence signal and gain of red fluorescence upon ferroptosis (right, arrows). Bottom images: loss of GFO fluorescence in ferroptotic cells is prevented by treatment with the osmoprotectant PEG3350. Times are relative values shown as hours:minutes. Ferroptosis was induced by treatment with 400 μM FAC and BSO.

FIGS. 5A-5B show that C′ dot nanoparticles induce ferroptosis that spreads through cell populations and kills prostate cancer cells in combination with Enzalutamide, an antiangrogen that targets androgens like testosterone and dihydrotestosterone (e.g., for hormone therapy).

FIG. 5A shows that C′ dot-treated cells undergo ferroptotic cell death that spreads through entire populations in a wave-like manner. The image shows nuclei of dead cells, pseudocolored to indicate the timing of cell death after treatment (from 19-24 hours).

FIG. 5B shows that prostate cancer-targeted PSMAi-C′ dots kill androgen-dependent prostate cancer cells (LNCaP) efficiently when combined with enzalutamide. Images show representative control and PSMAi-C′ dot+enzalutamide-treated LNCaP cells.

FIG. 6 shows that C′ dot ferroptotic induction inhibits in vivo prostate cancer growth. Male, LNCAP tumor-bearing mice, were intravenously (IV) administered saline vehicle, 60 μM PSMAi-PEG-Cy5-C′ dots, 60 μM αMSH-PEG-Cy5-C′ dots on days 0, 3, and 6 (200 μl/injection). Tumor growth was monitored over time use caliper measurements, and volumes were calculated using: Volume=(Long Axis×Short Axis²)/2. Total tumor volumes were normalized to Day 0 volumes and plotted using GraphPad Prism software.

FIGS. 7A-7C show that enzalutamide exposure increases PSMA expression in vitro and in vivo.

FIG. 7A shows a Western Blot of LNCAP prostate cancer cells that were continuously exposed to 10 μM enzalutamide in vitro for 15 days. At the conclusion of exposure, cells were collected and the expression levels of PSMA and AR were examined via Western blot.

FIG. 7B shows that, similar to results demonstrated in FIG. 7A, daily administration of enzalutamide (10 mg/kg/day; oral gavage) to mice bearing LNCAP xenografts also resulted in an increase in PSMA expression at Day 5.

FIG. 7C shows images of tumor sections that were collected from mice that were also used to evaluate PSMA expression using immunofluorescence staining. Staining for PSMA in LNCAP xenografts again supports an increase in PSMA expression at Day 5, demonstrated as an increase in fluorescence signal.

FIGS. 8A-8B show Western Blots indicating that exposure to enzalutamide increases PSMA expression in LNCAP-AR (PSMA+) but not PC-3 (PSMA−) control cell lines in vitro.

FIG. 8A shows a Western Blot of LNCAP-AR prostate cancer cells that were utilized as an anti-androgen (enzalutamide) resistant control line. Continuous exposure to 10 μM enzalutamide in vitro over the course of 15 days resulted in an time-dependent increase in PSMA expression, a result similar to observations in parental LNCAP cells.

FIG. 8B shows a Western Blot of PC-3 prostate cancer cells, which are negative for PSMA expression, that were utilized as a second control cell line. PC-3 cells exposed to 10 μM enzalutamide for 15 days in vitro demonstrate a lack of PSMA expression at all time points. Additionally, PC-3 cells are also negative for AR across all tested time points.

### DETAILED DESCRIPTION

Throughout the description, where compositions are described as having, including, or comprising specific components, or where methods are described as having, including, or comprising specific steps, it is contemplated that, additionally, there are compositions of the present invention that consist essentially of, or consist of, the recited components, and that there are methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

### Regulators of New Cell Activities Linked to Ferroptosis

It is presently discovered that ferroptosis occurs in a regulated manner and likely involves the presence of a plasma membrane pore. Current models for induction of necrosis through ferroptotic mechanisms involve damage through peroxidation to internal cellular membranes (e.g., ER) or to the plasma membrane, leading to cell death that is characterized by cell rupture. While other forms of regulated necrosis involve the assembly of pore structures that mediate the osmotic imbalances responsible for cell rupture, (for example the pore-forming Gasdermin D protein induces cell rupture during pyroptosis; MLKL during necroptosis), no such pore is known to participate in ferroptotic cell death. Rather, direct, unregulated membrane damage is thought to underlie ferroptotic cell rupture in response to iron accumulation and depletion of glutathione.

To investigate if the ferroptotic rupture of cells might be an event that is regulated by a pore, experiments were performed with osmo-protectants in the cell culture medium. Osmo-protectants are large osmolytes that when added to sufficient concentrations outside of cells (30 mM), buffer potential ion imbalances caused by pore formation that would otherwise lead to membrane rupture. The loss of ion and small molecule balances across the plasma membrane due to the opening of a pore would lead to hypo-osmotic stress due to the large molecules and organelles that cannot cross the intact membrane. Similar osmo-protectant experiments were used to demonstrate the presence of plasma membrane pores forming during the execution of pyroptosis and necroptosis.

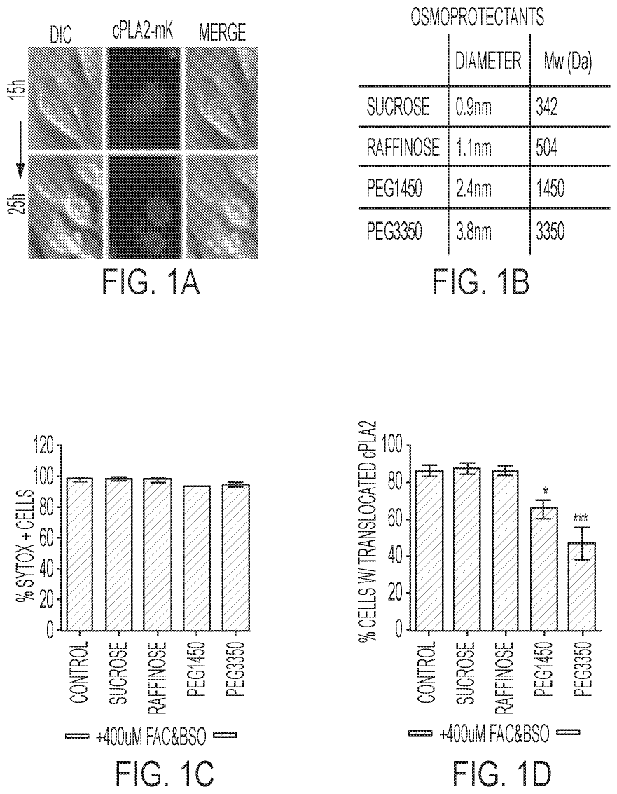

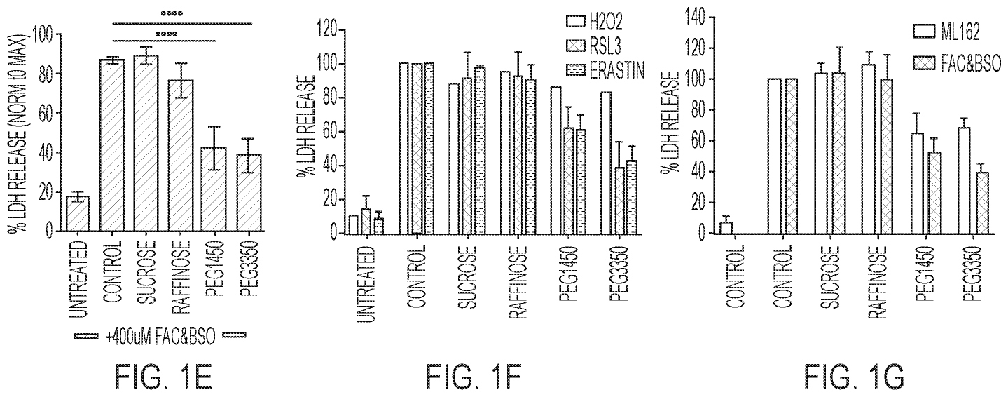

As shown in FIG. 1A, cell swelling (or osmotic imbalance) was observed during ferroptosis, indicated by translocation of a zebrafish cPLA2 enzyme to the nuclear membrane (a known indicator of cell swelling) prior to cell rupture. The use of osmo-protectants (FIG. 1B) of increasing sizes can indicate the presence of pores in the membrane that would be required for cell rupture. As shown in FIG. 1C, while the addition of osmo-protectant molecules to culture medium had no effect on the percentages of cells that died by ferroptosis, in response to treatment with 400 μM ferric ammonium citrates (FAC) and 400 μM of the glutathione synthesis-inhibiting compound buthionine sulfoximine (BSO) (indicated by positive staining of cells for the nucleic acid dye Sytox green), osmo-protectants did have a significant effect to block cell swelling (FIG. 1D) and also on the actual rupture of cells (FIG. 1E), indicated by quantification of the cytoplasmic enzyme lactate dehydrogenase in the medium, a commonly-used measure of necrosis. The largest-sized osmo-protectans (FIG. 1B), polyethylene glycol (PEG)3350 and PEG1450, significantly inhibited cell rupture, while the smaller raffinose and sucrose did not (FIG. 1E), demonstrating a narrow size cutoff to the effect of osmo-protectant addition, consistent with the presence of a regulated pore structure of particular size that regulates cell rupture during ferroptosis. In addition, ferroptosis induced by treatment with ferroptosis-inducing agents erastin, RSL3 (FIG. 1F), and MLN162 (FIG. 1G) also involved cell rupture that was inhibited by the addition of PEG1450 or PEG3350 to the medium, demonstrating that ferroptosis is generally regulated in a manner involving membrane pores. Necrotic cell death induced by treatment with hydrogen peroxide (H₂O₂) did not involve similar regulation (FIG. 1F). These data demonstrate the presence of a pore during ferroptosis, which resembles regulation during other necrotic forms of cell death such as (1) pyroptosis, which is involves a pore-forming protein called Gasdermin D, or (2) necroptosis, which involves a pore forming protein MLKL. As the knockout of the Gasdermin D gene (GsdmD) had no effect on ferroptotic cell rupture, and other Gasderin D family proteins were not observed to undergo cleavage (which would be indicative of their activation); and, treatment of cells with necrosulfonamide (NSA), an inhibitor of MLKL, did not affect ferroptotic cell rupture, ferroptosis is believed to involve a novel gene product that can be identified by screening.

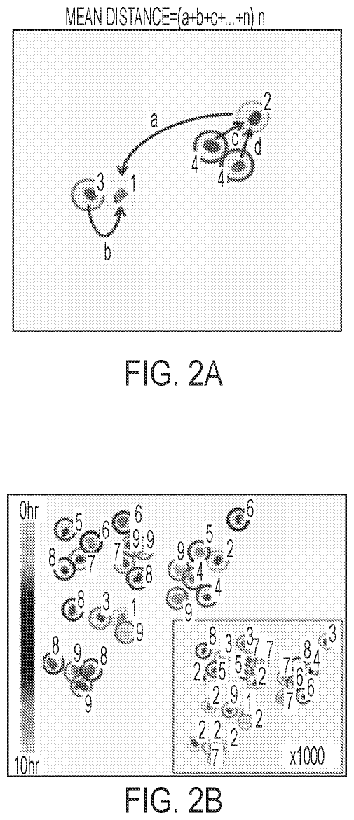

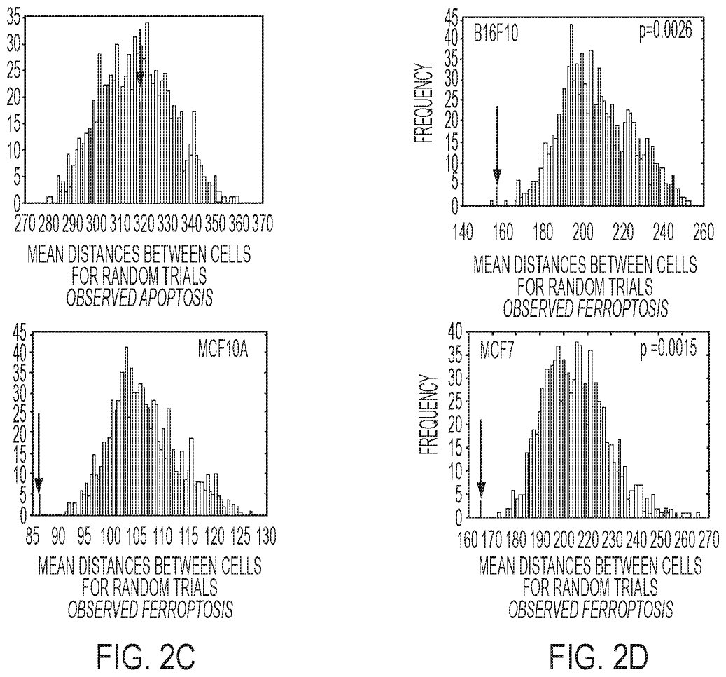

It was observed that ferroptosis involves the spreading of cell death between adjacent cells in cell culture in wave-like or synchronous patterns. While this activity may in part underlie the strong anti-cancer effects of ferroptosis induction that have been observed in experimental tumors in mice, how ferroptotic cell death spreads is unknown. To study cell death spreading, a computational analysis was designed that allows for quantification of the random or non-random nature of observed patterns of cell death in a population. As shown in FIGS. 2A-2D, this analysis utilizes time-lapse microscopic imaging of cell death, for cells cultured in the presence of Sytox green or Sytox orange dyes (or propidium iodide), which enter the nucleus of necrotic cells and emit green or red fluorescence (FIG. 2A). Computational analysis of the spatiotemporal patterns of cell death involves measuring the distance between successive cell deaths occurring over time in each microscopic field of view, to generate a mean distance between cell death events occurring over time (FIG. 2A). Wave-like or synchronous patterns will involve successive deaths spreading between neighboring cells, and thus the mean distance calculated for a wave-like pattern over time will be significantly smaller than random. In addition to the actual data set calculated for each field of view, randomized trials are then also performed 1000 times for each microscopic field, to generate a random set of possible mean distances that account for the particular arrangements of cells in each field (FIG. 2B). Comparison of observed mean distances to random trials of each field of view allows for the generation of p-values that express if observed patterns are significantly non-random (FIGS. 2C-2D). The generation of random means for each field of view, the quantification of actual observed means, and all statistical analyses are performed by custom MatLab software.

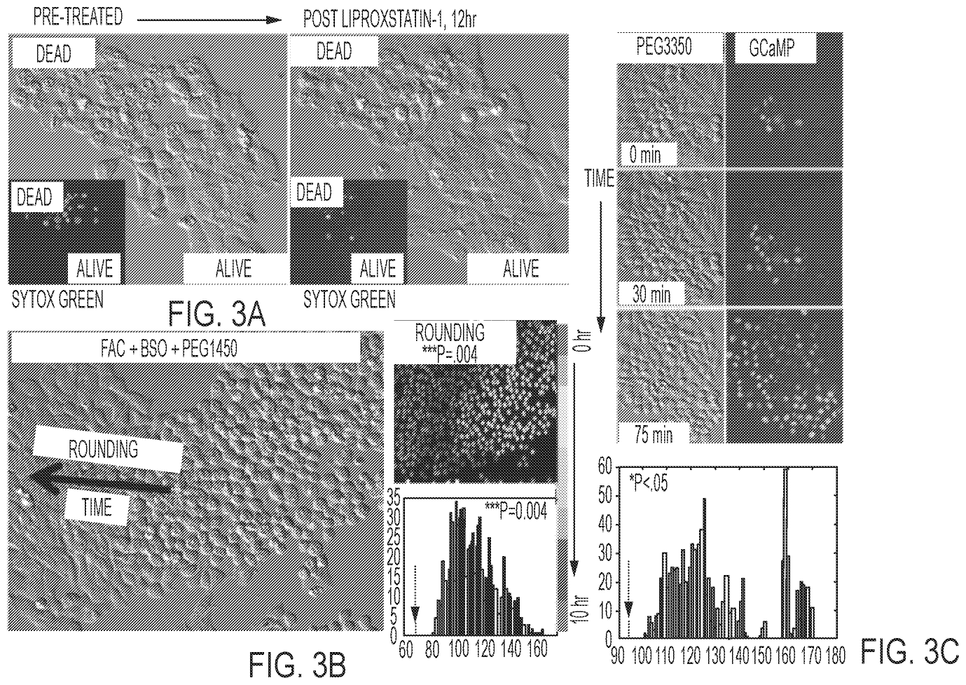

It was observed that ferroptosis involves a pore-forming activity and, also, a spreading activity. Treatment with ferroptosis inhibiting agent liproxstatin-1, a scavenger of lipid peroxides, disrupted wave-like spreading, even when added to culture after the initiation of ferroptosis and wave-like spreading (FIG. 3A). Similar results were obtained by treatment with the iron-chelating agent Desferoxamine (DFO), an inhibitor of ferroptosis. These results demonstrate that spreading of ferroptosis requires lipid peroxidation and iron. To examine if spreading requires cell rupture mediated by membrane pores, cells were treated with FAC and BSO (400 uM) to induce ferroptosis, and cell death patterns were computationally analyzed in the presence of PEG1450 or PEG3350 that inhibit cell rupture. As shown in FIG. 3B, while osmo-protectant addition inhibited cell rupture, it did not block a spatiotemporal pattern of spreading that involved cell rounding or swelling. This spreading pattern was analyzed computationally (as in FIGS. 2A-2D), and was found to occur in a significantly non-random manner (FIG. 3B; P=0.004). As cell rounding or swelling may be associated with calcium influx into cells, cells expressing the genetically-encoded calcium sensor GCaMP, which emits GFP fluorescence upon calcium exposure, were treated with FAC and BSO to induce ferroptosis in the presence of PEG3350. As shown in FIG. 3C, GCaMP-mediated green fluorescent signals were observed in cells upon cell rounding, and GCaMP fluorescence also spread through the cell population in a wave-like, non-random manner, as determined by using our computational analysis (FIG. 3C). Together these data demonstrate that ferroptosis is associated with a spreadable factor that requires lipid peroxidation and iron, and leads to wave-like propagation of cell swelling and calcium signaling upstream of cell rupture.

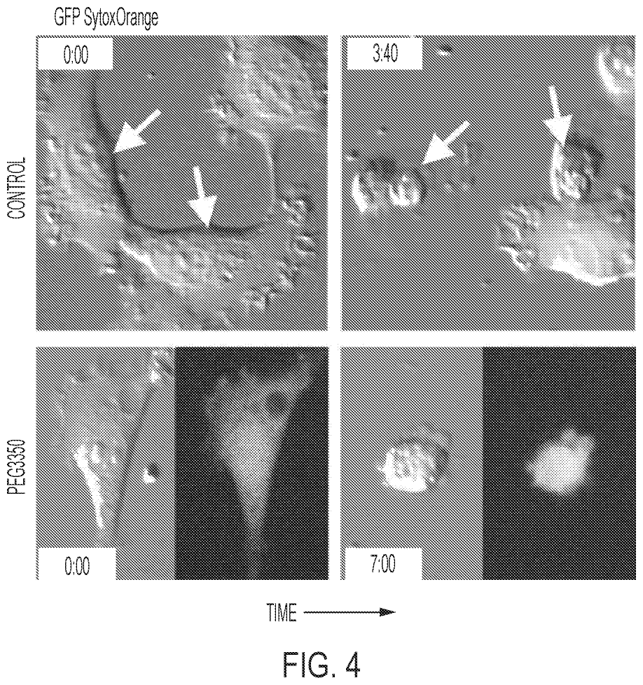

While these data demonstrate that ferroptosis involves pore-like regulation of cell rupture and an upstream spreading activity, the mechanisms that directly control these aspects are unknown. To address this issue, it is possible to identify genetic or chemical modulators of these ferroptosis parameters by high content, imaging-based screening of ferroptotic cell death occurring in response to treatment of cells with FAC and BSO. Screening may involve fluorescence time-lapse microscopy of cells expressing GFP and incubated in the presence of Sytox orange or propidium iodide (PI). As shown in FIG. 4, ferroptotic cell death results in the loss of GFP from cells and the gain of red fluorescent (Sytox orange or PI) due to cell rupture (FIG. 4). Treatment cells with the osmo-protectant PEG3350 inhibited the release of GFP, but not the acquisition of Sytox orange staining in response to FAC and BSO, demonstrating that GFP loss involves rupture, likely occurring downstream of a regulated pore that induces osmotic imbalance. Sytox orange staining could still occur by the dye entering through this pore, or by leaking across smaller holes in the plasma membrane. To identify regulators of ferroptosis (activators or inhibitors), it is possible to examine similar cells by time-lapse imaging, in the presence of genetic loss-of-function libraries (e.g. CRISPR or shRNA) or chemical libraries (e.g. FDA-approved drug library, Selleck chemicals) and treatment with FAC and BSO (or either agent alone), to quantify GFP fluorescence and Sytox orange or PI fluorescence patterns over time as ferroptosis occurs. Hits that inhibit the loss of GFP, and may or may not inhibit the acquisition of red fluorescence, will be further examined in follow-up analyses for effects on regulated pore formation, involving localization studies of candidate proteins and further loss-of-function analyses for other known ferroptosis parameters such as lipid peroxidation that is predicted to occur upstream. Ferroptosis induction in treated cultures can be demonstrated by inhibition of cell death through treatment with the known ferroptosis-inhibiting agent liproxstatin-1. In addition, the cell death spreading parameters can be quantified for each microscopic field of view to identify genetic or chemical hits that affect spreadability (by enhancing or inhibiting), leading to either random, or alternatively more synchronous or rapid, patterns of cell death (as per the computational analyses described in FIGS. 2A-2D). Potential hits affecting spreadability can be further examined for roles in pore formation, iron metabolism and trafficking, or glutathione production and redox biology in follow-up studies. For all validated screening hits, the effects of ferroptosis induction or inhibition can be further examined in murine cancer models where it is shown that ferroptosis induces an anti-cancer response.

### Imaging Systems and Methods

The analysis may make use of super-resolution microscopes to provide cellular, sub-cellular, organelle-level, and sub-organelle level resolutions. For example, a STORM/TIRF system (e.g., Nikon) with widefield FLIM and TIRF-FLIM (FLIM=Fluorescence Lifetime Imaging), ground state depletion (GSD) microscopy, direct stochastic optical reconstruction microscopy (dSTORM), stimulated emission and depletion (STED), and photoactivated localization microscopy (PALM), or an OMX Blaze 3D-SIM super-resolution microscope may be used.

Certain embodiments are directed to super-resolution tracking of drug delivery, metabolite delivery, radiotherapy, ferroptotic induction (e.g., by administration of nanoparticles), and the like, using optically-driven technologies (e.g., super-resolution microscopy). Various embodiments for which such tracking may be employed include therapeutic methods, combination therapies, and surgical procedures. For example, it is possible to monitor drug and/or substrate delivery and trafficking to subcellular compartments in human cancers (e.g., in perioperative settings, in situ and/or ex vivo). Drug/substrate delivery can be monitored at the level of specific organelles (e.g., lysosome, mitochondria, plasma membrane, and/or nucleus) and/or the cytosol, for assessment of treatment efficacy and/or fine-tuning treatment responses. Delivery efficiency and/or subcellular localization of the drug, nanoparticle, and/or other administered substrate may be assessed at a given point in time, or may be tracked over time, e.g., over a treatment period. Tissue sections from treated cancer specimens can also be examined ex-vivo for probe localization at subcellular and sub-organelle resolution by advanced techniques. For example, in certain embodiments, dual color super-resolution confocal imaging is performed on cells expressing a fluorescent marker of lysosomes and treated with a fluorescent nanoparticle of interest. The intracellular localization of single probes and small clusters of probes either to lysosomes, for instance, or to the surrounding cytosol, are determined and the percentages of co-localization patterns quantified. Cells treated over a time-course and with increasing concentrations are examined. If probes are observed outside of lysosomal membranes, further co-localization studies with additional organelle markers (i.e. mitochondria, endoplasmic reticulum, autophagosomes) are performed to identify additional sites of localization. These analyses can serve as a baseline for continued analysis utilizing probes bearing a range of surface chemistries and/or altered physicochemical properties. Tumor tissue section studies can be performed, for example, using thin Lamp1-GFP xenografted tumor sections (e.g., 2-10 μm) from mice previously treated with the probe of interest, and similar super-resolution microscopy studies performed to examine intralysosomal versus cytosolic localization of intracellular probes. For these studies, a series of frozen or fixed sections can be examined to define a protocol suitable for super-resolution microscopy.

In certain embodiments, a super-resolution microscope is employed. For example, a STORM/TIRF system (e.g., Nikon) with widefield-FLIM and TIRF-FLIM may be employed (FLIM=Fluorescence Lifetime Imaging). Various laser lines may be used for STORM (2D or 3D capabilities) and TIRF (e.g., 405, 488, 561, and 647 nm laser lines). A Lambert Instruments frequency domain LIFA module for FLIM can be used, either in widefield (LED) or laser (TIRF) mode (e.g., 445 and 514 nm lasers). A Photonics Instruments Micropoint laser may be used for photoablation, bleaching, and/or activation. DG5 may be used for widefield illumination. The system may also include a piezo x,y,z stage, an Andor DU-897 EMCCD camera, an Andor Neo sCMOS camera, a Tokai Hit environmental chamber, various objectives suitable for widefield and/or TIRF microscopy, and acquisition software (e.g., Elements acquisition software, Nikon).

Another example super-resolution microscope that may be employed includes an OMX Blaze 3D-SIM super-resolution microscope (Applied Precision). The microscope system may have, for example, 405, 445, 488, 514, and/or 568 nm lasers for 3D-SIM super-resolution imaging. The system may include a multi-line (e.g., 6-line) SSI module for ultra-rapid conventional imaging (e.g., to supplement super-resolution imaging). The system may further include, for example, a 100×/1.40 NA UPLSAPO oil objective (Olympus); multiple Evolve EMCCD cameras (Photometrics) for simultaneous or sequential acquisition (e.g., three cameras); and/or a heating chamber for live cell imaging.

In addition to localization, tracking, and delivery of nanoparticles, drugs, and/or other administered substances at the cellular, sub-cellular, and/or sub-organelle level, it is possible to simultaneously (or alternatively) monitor pH and/or metabolic species (e.g., oxygen (ROS) and/or glutathione) within cells and tissues, e.g., for real-time assessment of therapeutic efficacy. Nano-based sensors may be used to track cancer regression or recurrence in response to treatment. It is possible to detect changes in the cancer microenvironment that are linked to progression (e.g., decreased extracellular pH) or therapeutic response (e.g., increased lysosomal pH, decreased cellular pH, and/or increased cellular/lysosomal ROS). pH (or metabolite)-detecting sensor-treated cancer cells can be imaged by confocal or super-resolution microscopy to quantify subcellular or extracellular localization, as well as intensities to indicate changes in pH and/or accumulation of specific metabolites. Absolute pH quantifications can be made based on standard curves generated by bathing cells in pH-adjusted buffers. The effects of induction of cell death (e.g. apoptosis) on intracellular or lysosomal pH can be determined in culture, prior to studies designed to detect the effects of therapeutic approaches on experimental xenografted tumors using imaging of thin fixed or frozen sections. Therapeutic approaches can be examined in combination with pH sensing. In certain embodiments, the extent of probe internalization within cancer cells is determined in tumor sections, as extracellular-localized probes are predicted to exhibit markedly increased pH profiles as compared to those within lysosomes. Relative changes in extracellular and intracellular pH in response to a variety of treatments can also be determined. In vitro studies can serve to inform the application of sensor technologies in vivo. A functional camera system can provide complementary real-time assessments of pH, oxygenation status, and small-vessel perfusion. The ability to utilize a range of wavelengths spanning ˜400-1000 nm allows for the measurement of different spectral absorption and emission profiles defining proteins, metabolic species, or the optical properties of externally administered probes. Hemoglobin, for instance, has different spectral characteristics in the NIR than deoxyhemoglobin. The functional camera system can discriminate these spectral differences over very discrete bandwidths, allowing spatial spectrometry to be performed. This set-up can also be applied to address key biological questions for a variety of tissue types and chromophores.

For example, in certain embodiments, metabolic imaging of cancer progression and therapy is performed by employing nanosensor delivery. Drug treatment responses and cancer progression are imaged by delivery of nanoparticles with sensor capability for pH and reactive oxygen species (ROS). Particle-intrinsic and drug conjugation-based therapeutics can be imaged in real time for assessment of efficacy, and nano-based sensors may be used to track cancer regression or recurrence in response to treatment. Particles with an engineered capability to sense changes in cellular or extracellular pH or ROS can be used to track changes in the cancer microenvironment.

In certain embodiments, dual color super-resolution confocal imaging can be performed on cells (e.g., M21 melanoma cells) expressing a fluorescent marker of lysosomes (Lamp1-GFP) and treated with Cy5-fluorescent C′ dot nanoparticles. The intracellular localization of single nanoparticles and small nanoparticle clusters either to the lysosome lumen or to the cytosol outside of the lysosomal membrane, can be determined and the percentages of co-localization patterns quantified.

Cells treated over a time-course and with increasing concentrations can be examined, including up to 15 μM, a concentration that causes significant intrinsic cell death-inducing capacity of C′ dots linked to the lysosomal delivery of iron (for example, as described by Bradbury et al., International (PCT) Patent Application No. PCT/US2016/034351, “Methods of Treatment Using Ultrasmall Nanoparticles to Induce Cell Death of Nutrient-Deprived Cancer Cells Via Ferroptosis,” the disclosure of which is hereby incorporated by reference in its entirety).

### Example Therapeutic Methods Involving Ferroptotic Induction

A ferroptotic induction step may be used in combination with immunotherapy and/or small molecule drugs to overcome chemo/immuno resistance mechanisms observed in current treatment therapies. For example, hormone therapy can be administered in combination with ferroptotic induction. Hormone therapies include (i) treatments to lower androgen levels (e.g., Orchiectomy (surgical castration), luteinizing hormone-releasing hormone (LHRH) agonists, LHRH antagonists (e.g., Degarelix (Firmagon), CYP17 inhibitors, and/or Abiraterone (Zytiga)), (ii) treatments to stop androgens from working (e.g., anti-androgens), and/or (iii) other androgen-suppressing drugs (e.g., estrogens, ketoconazole).

In certain embodiments, hormone therapy is used in a select group of patients where high levels of a biomarker are expressed within the cancer cells and/or tumor tissue (e.g., PSMA expression in prostate cancer). Combination therapy helps to provide an alternative method of treatment for subjects who have developed resistance to hormone therapy.

Ferroptosis induction has been found to involve the spreading of cell death through cancer cell populations in a wave-like manner, whereby death spreads from treated to untreated cells. In certain embodiments, this propagating feature of ferroptotic cell death may offer high therapeutic potential for the treatment of cancer, as cell death induction could spread in even a small population of cancer cells to achieve a more complete kill (including cancer stem-like cells), than the induction of other death forms (i.e., apoptosis). “Self-therapeutic” ferroptosis-inducing particles, as well as other non-particle ferroptosis-inducing agents, may be used as part of a combination treatment paradigm, along with immune checkpoint blocking antibodies and selective inhibitors of myeloid cell targets, for example, to overcome mechanisms of immune resistance in melanoma patients. Furthermore, immune modulators may be delivered and released to regulate the TME, including tumor-associated macrophages (TAMs) and effector cells, and/or improving responses to T-cell checkpoint immunotherapy. Nanoparticles can be used to selectively target pathways known to influence differentiation and survival of macrophages, as well as their activation or polarization state, such as colony stimulating factor-1 (CSF-1). Tumor models sensitive to TME regulation via this pathway can be targeted with CSF-1 small molecule inhibitors, such as BLZ945. Additional targeted inhibitors can be used in combination to disrupt tumor cell signaling via alternative pathways.

In certain embodiments, the nanoparticle acts as an immunomodulator (mechanism is unknown), in addition to (or as part of) inducing ferroptosis. Other immunomodulators may be used in combination with the nanoparticle.

Thus, in certain embodiments, ferroptotic induction is performed in combination with any one or more of immunotherapy, hormonal therapy, chemotherapy, radiotherapy (e.g., alpha-, beta-, or gamma-emitters) and/or small molecule drug administration. These treatment combinations are operative through various mechanisms to yield synergistic or additive responses. In addition, the contribution of ferroptosis to responses elicited solely with small molecule inhibitors (or other therapeutic options) can be assessed. In particular, in certain embodiments, ferroptotic induction may be particularly helpful in overcoming resistance that is seen following treatment with a particular agent or class of agents over a period of time, thereby prolonging and/or otherwise enhancing the effectiveness of a given course of treatment. In certain embodiments, methods and/or compositions described in International (PCT) Patent Application No. PCT/US2016/034351, “Methods of Treatment Using Ultrasmall Nanoparticles to Induce Cell Death of Nutrient-Deprived Cancer Cells Via Ferroptosis”, incorporated by reference herein in its entirety, may be used, for example, in the ferroptotic induction step in the methods described herein. Furthermore, in certain embodiments, ferroptotic induction is assessed and/or monitored using a super-resolution microscope described herein and/or nanoprobes described herein, e.g., to assess mechanisms associated with derangements of the tumor microenvironment (TME), to reprogram the TME, and/or to adjust the therapy. Examples are presented below for prostate cancer and melanoma using C′ dots.

Various embodiments described herein utilize ultrasmall FDA-IND approved nanoparticles, such as C and C′ dots. Various embodiments described herein demonstrate their adaptation for drug delivery applications and detail cell biological analyses examining (1) how cells and xenograft models respond to melanoma-targeting C′ dot (e.g., α-MSH-PEG-C′ dots) treatment over a range of concentrations and times (e.g., days to weeks), and (2) whether cellular pathways are affected by particle ingestion were performed. Further description of these embodiments is included in Bradbury et al. U.S. Publication No. US 2014/0248210 A1 entitled “Multimodal Silica-Based Nanoparticles”, the contents of which is hereby incorporated by reference in its entirety.

In International (PCT) Patent Application No. PCT/US2016/034351, “Methods of Treatment Using Ultrasmall Nanoparticles to Induce Cell Death of Nutrient-Deprived Cancer Cells Via Ferroptosis”, incorporated by reference herein in its entirety, it is demonstrated that a combination of treatment of cells with α-MSH-PEG-C′ dots and starvation of amino acids synergize to induce the cell death program ferroptosis. For the present data, while PSMAi-PEG-C′ dots induced a modest ferroptotic induction, their combination with enzalutamide in vitro was synergistic in PSMA-expressing LNCaP lines. In vivo significant tumor volume reductions were sustained with both MC1-R-targeting and PSMA-targeting C′ dots. Moreover, it is demonstrated herein that concentration- and time-dependent treatment effects on cells using sub-10 nm diameter fluorescent (Cy5 dye-containing) silica nanoparticles, (e.g., C′ dots) adapted with melanoma-targeting peptides. International (PCT) Patent Application No. PCT/US2016/034351, the contents of which is hereby incorporated by reference in its entirety, describes how high concentrations of ultrasmall nanoparticles (e.g., less than 10 nm in diameter, e.g., C dots or C′ dots) induce cell death by the mechanism ferroptosis, which involves iron, reactive oxygen species, and a synchronous mode of cell death execution. In certain embodiments, the high concentration is a local concentration within a range from 0.18 μM to 1.8 μM in cancer cells and/or tumor tissue of a subject (wherein this range is an estimate based on the mouse studies described herein). In certain embodiments, the high concentration is a local concentration in the cancer cells and/or tumor tissue of at least 0.18 μM, at least 0.3 μM, at least 0.4 μM, at least 0.5 μM, or at least 0.6 μM; e.g., wherein the nanoparticles are silica-based, e.g., wherein the nanoparticles are C dots or C′ dots. In certain embodiments, the local concentration is dependent on tumor type and/or the subject.

High concentrations of these particles were generally well tolerated in both non-cancer and cancer cells cultured in nutrient-replete media. The combination of particle treatment and metabolic (e.g., amino acid) deprivation synergized to kill cancer cells at high rates. Without having to be bound by theory, ingested nanoparticles localize to lysosome networks, but do not inhibit lysosome function, and nanoparticle-induced death occurs independently of the autophagy pathway.

To determine whether these effects extended to nutrient depleted conditions in vivo, xenografts were generated from particle-exposed cancer cells or treated intravenously using a particle multi-dosing strategy. To this end, concentration-dependent sustained growth inhibition was observed, and suppression of tumor growth kinetics coupled with a partial tumor regression occurred. Thus, these data demonstrated that ultrasmall, surface-functionalized silica-based nanoparticles, employed under high concentration and nutrient-deprived conditions, induced cell death by the mechanism ferroptosis.

### Nanoparticle

In certain embodiments of any of the methods described herein, the nanoparticle comprises silica, polymer (e.g., poly(lactic-co-glycolic acid) (PLGA)), biologics (e.g., protein carriers), and/or metal (e.g., gold, iron). In certain embodiments, the nanoparticle is a “C dot” as described in U.S. Publication No. 2013/0039848 A1 by Bradbury et al., which is hereby incorporated by reference.

In certain embodiments, the nanoparticle is spherical. In certain embodiments, the nanoparticle is non-spherical. In certain embodiments, the nanoparticle is or comprises a material selected from the group consisting of metal/semi-metal/non-metals, metal/semi-metal/non-metal-oxides, -sulfides, -carbides, -nitrides, liposomes, semiconductors, and/or combinations thereof. In certain embodiments, the metal is selected from the group consisting of gold, silver, copper, and/or combinations thereof.

In certain embodiments, the nanoparticle is a nanoparticle as described in U.S. Pat. No. 8,409,876 entitled “Fluorescent Silica-Based Nanoparticles” filed on Oct. 14, 2009, U.S. Pat. No. 8,298,677 entitled “Fluorescent Silica-Based Nanoparticles” filed on May 2, 2006, U.S. Pat. No. 8,084,001 entitled “Photoluminescent Silica-Based Sensors and Methods of Use” filed on May 2, 2006, U.S. Pat. No. 8,961,825 entitled “Fluorescent Silica Nanoparticles Through Silica Densification” filed on Apr. 27, 2012, U.S. Patent Publication No. US 2015-0366995 A1 entitled “Mesoporous Oxide Nanoparticles and Methods of Making and Using Same” filed on Dec. 22, 2014, U.S. Patent Publication No. US 2016-0018404 A1 entitled “Multilayer Fluorescent Nanoparticles and Methods of Making and Using Same” filed on Aug. 19, 2015, U.S. Patent Publication No. US 2018-0133346 A1 entitled “Ultrasmall Nanoparticles and Methods of Making and Using Same” filed on Nov. 2, 2017, and International Patent Application No. PCT/US18/33755 entitled “Functionalized Nanoparticles and Methods of Making Same” filed on May 21, 2018, the contents of each of which are hereby incorporated by reference in their entireties.

In certain embodiments, one or more nanoparticles are selected from the photoswitchable nanoparticles described by Kohle et al., “Sulfur- or Heavy Atom-Containing Nanoparticles, Methods or Making the Same, and Uses Thereof,” in International Application No. PCT/US18/26980 filed on Apr. 10, 2018, the photoluminescent silica-based sensors described by Burns et al. “Photoluminescent Silica-Based Sensors and Methods of Use” in U.S. Pat. No. 8,084,001 B 1, and/or the nanoparticles described by Bradbury et al., “Ultrasmall Nanoparticles Labeled with Zirconium-89 and Methods Thereof,” International Patent Application No. PCT/US18/33098, filed on May 17, 2018. In certain embodiments, the nanoparticle is a modification or combination of any of such compositions.

In certain embodiments, the present disclosure also describes nanoparticle compositions that comprise a PDT-active moiety (e.g., methylene blue) associated (e.g., covalently bound, e.g., non-covalently bound) with silica-based nanoparticles. In certain embodiments, the nanoparticle compositions are those described by Kohle et al. in U.S. Provisional Application No. 62/666,086 entitled “Functionalized Sub-10 nm Silica Nanophotosensitizers,” filed on May 4, 2018, the disclosure of which is hereby incorporated by reference in its entirety.

The nanoparticle may comprise metal/semi-metal/non-metal oxides including silica (SiO₂), titania (TiO₂), alumina (Al₂O₃), zirconia (ZᵣO2), germania (GeO₂), tantalum pentoxide (Ta₂O₅), NbO₂, and/or non-oxides including metal/semi-metal/non-metal borides, carbides, sulfide and nitrides, such as titanium and its combinations (Ti, TiB₂, TiC, TiN).

The nanoparticle may comprise one or more polymers, e.g., one or more polymers that have been approved for use in humans by the U.S. Food and Drug Administration (FDA) under 21 C.F.R. § 177.2600, including, but not limited to, polyesters (e.g., polylactic acid, poly(lactic-co-glycolic acid), polycaprolactone, polyvalerolactone, poly(1,3-dioxan-2-one)); polyanhydrides (e.g., poly(sebacic anhydride)); polyethers (e.g., polyethylene glycol); polyurethanes; polymethacrylates; polyacrylates; polycyanoacrylates; copolymers of PEG and poly(ethylene oxide) (PEO).

The nanoparticle may comprise one or more degradable polymers, for example, certain polyesters, polyanhydrides, polyorthoesters, polyphosphazenes, polyphosphoesters, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Another exemplary degradable polymer is poly (beta-amino esters), which may be suitable for use in accordance with the present application.

In certain embodiments, a nanoparticle can have or be modified to have one or more functional groups. Such functional groups (within or on the surface of a nanoparticle) can be used for association with any agents (e.g., detectable entities, targeting entities, therapeutic entities, or PEG). In addition to changing the surface charge by introducing or modifying surface functionality, the introduction of different functional groups allows the conjugation of linkers (e.g., (cleavable or (bio-)degradable) polymers such as, but not limited to, polyethylene glycol, polypropylene glycol, PLGA), targeting/homing agents, and/or combinations thereof. In certain embodiments, the nanoparticle comprises one or more targeting ligands as described in International Patent Application No. PCT/US17/63641, “Inhibitor-Functionalized Ultrasmall Nanoparticles and Methods Thereof,” filed Nov. 29, 2017, published as WO/2018/102372, which is incorporated herein by reference in its entirety.

In certain embodiments, the nanoparticle comprises one or more targeting ligands (or moieties) (e.g., attached thereto), such as, but not limited to, small molecules (e.g., folates, dyes, etc), aptamers (e.g., A10, AS1411), polysaccharides, small biomolecules (e.g., folic acid, galactose, bisphosphonate, biotin), oligonucleotides, and/or proteins (e.g., (poly)peptides (e.g., αMSH, RGD, octreotide, AP peptide, epidermal growth factor, chlorotoxin, transferrin, etc), antibodies, antibody fragments, proteins). In certain embodiments, the nanoparticle comprises one or more contrast/imaging agents (e.g., fluorescent dyes, (chelated) radioisotopes (SPECT, PET), MR-active agents, CT-agents), and/or therapeutic agents (e.g., small molecule drugs, therapeutic (poly)peptides, therapeutic antibodies, radioisotopes, chelated radioisotopes). In certain embodiments, the radioisotope used as a contrast/imaging agent and/or therapeutic agent comprises any one or more of ^{99m}Tc, ⁶⁴Cu, ⁶⁷Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹¹C, ¹³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, ¹⁴⁰La, ¹⁹⁸Au, ¹⁹⁹Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, ⁸⁹Zr, ²²⁵Ac, and ¹⁹²Ir.

In certain embodiments, PET (Positron Emission Tomography) tracers are used as imaging agents. In certain embodiments, PET tracers comprise Zr⁸⁹, Cu⁶⁴, ²²⁵Ac, and/or ¹⁸F. In certain embodiments, the PET tracer comprises fluorodeoxyglucose. In certain embodiments, the nanoparticle includes these and/or other radiolabels. In certain embodiments, the one or more targeting ligands (or moieties) can be of the same type, or can be different species.

In certain embodiments, the nanoparticle comprises one or more fluorophores. Fluorophores comprise fluorochromes, fluorochrome quencher molecules, any organic or inorganic dyes, metal chelates, or any fluorescent enzyme substrates, including protease activatable enzyme substrates. In certain embodiments, fluorophores comprise long chain carbophilic cyanines. In other embodiments, fluorophores comprise DiI, DiR, DiD, and the like. Fluorochromes comprise far red, and near infrared fluorochromes (NIRF). Fluorochromes include but are not limited to a carbocyanine and indocyanine fluorochromes. In certain embodiments, imaging agents comprise commercially available fluorochromes including, but not limited to Cy5.5, Cy5 and Cy7 (GE Healthcare); AlexaFlour660, AlexaFlour680, AlexaFluor750, and AlexaFluor790 (Invitrogen); VivoTag680, VivoTag-S680, and VivoTag-S750 (VisEn Medical); Dy677, Dy682, Dy752 and Dy780 (Dyomics); DyLight547, DyLight647 (Pierce); HiLyte Fluor 647, HiLyte Fluor 680, and HiLyte Fluor 750 (AnaSpec); IRDye 800CW, IRDye 800RS, and IRDye 700DX (Li-Cor); and ADS780WS, ADS830WS, and ADS832WS (American Dye Source) and Kodak X-SIGHT 650, Kodak X-SIGHT 691, Kodak X-SIGHT 751 (Carestream Health).

In certain embodiments, the nanoparticle comprises (e.g., has attached) one or more targeting ligands, e.g., for targeting cancer tissue/cells of interest.

Cancers that may be treated include, for example, prostate cancer, breast cancer, testicular cancer, cervical cancer, lung cancer, colon cancer, bone cancer, glioma, glioblastoma, multiple myeloma, sarcoma, small cell carcinoma, melanoma, renal cancer, liver cancer, head and neck cancer, esophageal cancer, thyroid cancer, lymphoma, pancreatic (e.g., BxPC3), lung (e.g., H1650), and/or leukemia.

In certain embodiments, the nanoparticle comprises a therapeutic agent, e.g., a drug (e.g., a chemotherapy drug) and/or a therapeutic radioisotope. As used herein, “therapeutic agent” refers to any agent that has a therapeutic effect and/or elicits a desired biological and/or pharmacological effect, when administered to a subject.

In certain embodiments, e.g., where combinational therapy is used, an embodiment therapeutic method includes administration of the nanoparticle and administration of one or more drugs (e.g., either separately, or conjugated to the nanoparticle), e.g., one or more chemotherapy drugs, such as gefitinib, sorafenib, paclitaxel, docetaxel, MEK162, etoposide, lapatinib, nilotinib, crizotinib, fulvestrant, vemurafenib, bexorotene, and/or camptotecin.

The surface chemistry, uniformity of coating (where there is a coating), surface charge, composition, concentration, frequency of administration, shape, and/or size of the nanoparticle can be adjusted to produce a desired therapeutic effect, e.g., ferroptosis of cancer cells.

It should be understood that use of the singular “nanoparticle” herein may mean multiple discrete particles of a particular type. Similarly, use of the plural “nanoparticles” herein may mean multiple discrete particles of a particular type, or multiple types of particles, depending on context.

In certain embodiments, the nanoparticles have an average diameter no greater than about 50 nm (e.g., no greater than about 40 nm, e.g., no greater than about 30 nm, e.g., no greater than about 25 nm, e.g., no greater than about 20 nm, e.g., no greater than about 15 nm, e.g., no greater than about 10 nm, e.g., no greater than about 8 nm). In certain embodiments, the nanoparticles have an average diameter no greater than 20 nm. In certain embodiments, the nanoparticles have an average diameter from about 5 nm to about 7 nm (e.g., about 6 nm).

In certain embodiments, nanoparticle drug conjugates (NDCs) are used for drug delivery applications. Detail on NDCs are described, for example, in International publication WO 2015/183882 A1, the content of which is hereby incorporated by reference it its entirety.

### Prostate Cancer (PC)-Functionalized Nanoparticles for Ferroptotic Induction

The present disclosure describes methods of treatment (e.g., combination treatment) by ferroptotic induction, as well as agents comprising prostate cancer (PC)-targeting nanoparticles (e.g., PC-targeting dots (C′ dots)). In certain embodiments, the described platform provides improved metastatic disease assessment and surgical treatment of PC by (1) promoting multivalent interactions with receptor targets that enhance potency and target-to-background ratios (contrast); and (2) exploiting its superior photophysical properties, alongside device technologies, to maximize detection sensitivity.

Ferroptotic inducing agents such as the Prostate Cancer (PC)-functionalized nanoparticles described herein offer at least the following advantages compared to alternative technologies: (1) an “all-in-one” dual-modality and clinically-translatable inhibitor (e.g., PSMA inhibitor, e.g., GRPr inhibitor)-targeting platform for perioperative management of PC; (2) utilization of spectrally-distinct PC-targeting C′ dots and fluorescence-based multiplexing strategies for real-time evaluation of multiple molecular cancer phenotypes; (3) qualification of reliable staging biomarkers targeting different biological processes for direct clinical validation; (4) characterization of inhibitor expression levels for new metastatic PC subclones and human prostate organoid-based models that may more faithfully reproduce human disease; (5) efficient optimization of new surface designs for renally-clearable PC-targeted C′ dots which overcome high non-specific uptake in radiosensitive organs (e.g., kidney, salivary glands), where such non-specific uptake has limited radiotherapeutic dosing and treatment efficacy; (6) use of particle-encapsulated NIR dyes to obviate attendant losses in bioactivity seen with NIR dye-conjugated inhibitor, the latter precluding NIR-driven optical applications; and (7) chemical adaptation of linker-peptide chemistries prior to C′ dot attachment to preserve pharmacophore activity while enhancing radiolabeling and tumor-targeting efficiencies.

Commercially available PSMAi-HBED-CC compounds are not compatible for conjugation to nanotherapeutic delivery systems. All reported studies evaluating PSMA inhibitor-metal chelator constructs have thus far focused on the use of the free compound. Preclinical studies have shown the PSMA inhibitor to be more effective (e.g., enhanced binding and cellular uptake) when coupled to certain types of metal chelators, than when used alone. While the PSMA inhibitor alone has been used on macromolecules for PSMA targeting, the development of PSMA inhibitor-metal chelator constructs, for example, PSMAi-HBED-CC analogs, compatible for conjugation onto a macromolecular entity have not been reported. Described herein are conjugates comprising a PSMA inhibitor and metal chelator that are covalently attached to a macromolecule (e.g., a nanoparticle, a polymer, a protein). Such conjugates may exhibit enhancements in binding and cell uptake properties (due to multivalency) and pharmacokinetics (due to increased molecular weight or size) over the free, unbound PSMA inhibitor/chelator construct. For example, PSMA inhibitor displayed on a macromolecule (e.g., nanoparticle) surface has reduced kidney uptake compared with PSMA inhibitor constructs alone.

Also described herein is the development of conjugates where constructs containing a PSMA inhibitor and metal chelator are covalently attached to a macromolecule (e.g., a nanoparticle, a polymer, a protein). Such conjugates may exhibit enhancements in binding and cell uptake properties (e.g., due to multivalency) and pharmacokinetics (e.g., due to increased molecular weight or size) over the free, unbound PSMA inhibitor/chelator construct. For example, PSMA inhibitor displayed on a macromolecule (e.g., nanoparticle) surface has reduced kidney uptake compared with free, unbound PSMA inhibitor constructs.

Details of various embodiments applicable to the systems and methods described herein are also provided in, for example, PCT/US14/30401 (WO 2014/145606) by Bradbury et al., PCT/US16/26434 (“Nanoparticle Immunoconjugates”, filed Apr. 7, 2016) by Bradbury et al., PCT/US14/73053 (WO2015/103420) by Bradbury et al., PCT/US15/65816 (WO 2016/100340) by Bradbury et al., PCT/US16/34351 (“Methods and Treatment Using Ultrasmall Nanoparticles to Induce Cell Death of Nutrient-Deprived Cancer Cells via Ferroptosis”, filed May 26, 2016) by Bradbury et al., U.S. 62/267,676 (“Compositions Comprising Cyclic Peptides, and Use of Same for Visual Differentiation of Nerve Tissue During Surgical Procedures” filed Dec. 15, 2015) by Bradbury et al., U.S. 62/330,029 (“Compositions and Methods for Targeted Particle Penetration, Distribution, and Response in Malignant Brain Tumors,” filed Apr. 29, 2016) by Bradbury et al., U.S. Ser. No. 14/588,066 “Systems, methods, and apparatus for multichannel imaging of fluorescent sources in real time” by Bradbury et al., and U.S. 62/349,538 (“Imaging Systems and Methods for Lymph Node Differentiation and/or Nerve Differentiation, e.g., for Intraoperative Visualization,” filed Jun. 13, 2016) by Bradbury et al., the contents of which are hereby incorporated by reference in their entireties. In certain embodiments, conjugates of the present disclosure are an agent comprising a targeting peptide/chelator construct covalently attached to a macromolecule. In certain embodiments, the targeting peptide comprises a prostate specific membrane antigen inhibitor (PSMAi). In certain embodiments, the targeting peptide comprises a bombesin/gastrin-releasing peptide receptor ligand (GRP).

In certain embodiments, PSMAi conjugates of the present disclosure are of the formula:

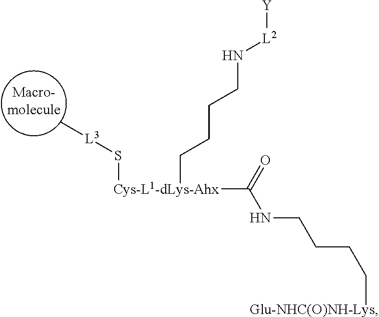

wherein:
L¹ is a peptidic fragment comprising from 1 to about 10 natural or unnatural amino acid residues, or an optionally substituted, bivalent, C₁₋₂₀ saturated or unsaturated, straight or branched, hydrocarbon chain, wherein one or more methylene units of the hydrocarbon chain are optionally and independently replaced by —CHOH—, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, or —C(═NR)—;
L² is an optionally substituted, bivalent, C₁₋₁₀ saturated or unsaturated, straight or branched, hydrocarbon chain, wherein one or more methylene units of the hydrocarbon chain are optionally and independently replaced by -Cy-, —CHOH—, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, or —C(═NR)—;
L³ is a covalent bond or a crosslinker derived from a bifunctional crosslinking reagent capable of conjugating a reactive moiety on the (PSMAi)/chelator construct with a moiety of the macromolecule;
each -Cy-is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Y is a chelator moiety; and
R is hydrogen, C₁₋₆ alkyl, or a nitrogen protecting group;
   wherein each amino acid residue, unless otherwise indicated, may be protected or unprotected on its terminus and/or side chain group.

It will be appreciated that throughout this disclosure, where a macromolecule (either generically or specifically) is drawn schematically as part of a conjugate, e.g.

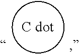

such schematic encompasses a suitable linking moiety between the macromolecule and its depicted attachment to the remainder of the conjugate.

In certain embodiments, a bombesin/gastrin-releasing peptide receptor ligand (GRP)/chelator construct comprises a peptide of the sequence:

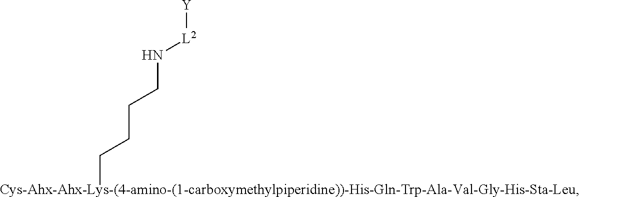

wherein,
L² is an optionally substituted, bivalent, C₁₋₁₀ saturated or unsaturated, straight or branched, hydrocarbon chain, wherein one or more methylene units of the hydrocarbon chain are optionally and independently replaced by -Cy-, —CHOH—, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, or —C(═NR)—;
each -Cy-is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; Y is a chelator moiety; and R is hydrogen, C₁₋₆ alkyl, or a nitrogen protecting group;
   wherein each amino acid residue, unless otherwise indicated, may be protected or unprotected on its terminus and/or side chain group.

In some embodiments, a bombesin/gastrin-releasing peptide receptor ligand (GRP)/chelator construct is covalently attached via the depicted cysteine residue to a macromolecule through a crosslinker, L³, wherein L³ is a covalent bond or a crosslinker derived from a bifunctional crosslinking reagent capable of conjugating a reactive moiety on the bombesin/gastrin-releasing peptide receptor ligand (GRP)/chelator construct with a reactive moiety of the macromolecule.

It will be appreciated that throughout this disclosure, unless otherwise specified, amino acid side chain groups or termini are optionally protected with a suitable protecting group.

In some embodiments, L¹ is a peptidic fragment comprising 1, 2, 3, 4, 5, 6, 7, 8, 9, or 10 natural or unnatural amino acid residues. In some embodiments, L¹ is a peptidic fragment comprising 1, 2, 3, 4, or 5 natural or unnatural amino acid residues. In some embodiments, L¹ is a peptidic fragment comprising 1, 2, or 3 natural or unnatural amino acid residues. In some embodiments, L¹ is a peptidic fragment comprising 2 unnatural amino acid residues. In some embodiments, L¹ comprises one or two units of 6-aminohexanoic acid (Ahx). In some embodiments, L¹ is -Ahx-Ahx-.

In some embodiments, L¹ is an optionally substituted C₁₋₁₀ saturated or unsaturated, straight or branched, hydrocarbon chain, wherein one or more methylene units of the hydrocarbon chain are optionally and independently replaced by —CHOH—, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, or —C(═NR)—. In some embodiments, L¹ is a C₁₋₁₀ saturated or unsaturated, straight or branched, hydrocarbon chain, wherein one or more methylene units of the hydrocarbon chain are optionally and independently replaced by —NR—, —O—, or —C(O)—. In some embodiments, L¹ comprises one or more units of ethylene glycol. In certain embodiments, L¹ comprises one or more units of —(CH₂CH₂O)— or —(OCH₂CH₂)—.

In certain embodiments, L² is a C₁₋₆ saturated or unsaturated, straight or branched, hydrocarbon chain, wherein one or more methylene units of the hydrocarbon chain are optionally and independently replaced by -Cy-, —NR—, —N(R)C(O)—, —C(O)N(R)—, —O—, —C(O)—, —OC(O)—, or —C(O)O—. In certain embodiments, L² is a C₁₋₃ saturated or unsaturated, straight or branched, hydrocarbon chain, wherein one or more methylene units of the hydrocarbon chain are optionally and independently replaced by -Cy-, —NR—, —N(R)C(O)—, —C(O)N(R)—, —O—, —C(O)—, —OC(O)—, or —C(O)O—. In certain embodiments, L² is a C₁₋₃ saturated or unsaturated, straight or branched, hydrocarbon chain, wherein one, two, or three, methylene units of the hydrocarbon chain are optionally and independently replaced by -Cy-, —NR—, or —C(O)—. In some embodiments, L² is —C(O)— or —C(O)NH-Cy-.

One of ordinary skill in the art will be familiar with a multitude of suitable crosslinking reagents for use in accordance with the provided methods. Such suitable crosslinking reagents are described in Hermanson, G. T. (2008). Bioconjugate Techniques. 2nd edition, Academic Press, New York. In certain embodiments, a crosslinking reagent is a heterobifunctional reagent. In certain embodiments, a crosslinking reagent is a homobifunctional reagent. In some embodiments, a bifunctional crosslinking reagent is selected from
i) maleimides (Bis-Maleimidoethane, 1,4-bismaleimidobutane, bismaleimidohexane, Tris[2-maleimidoethyl]amine, 1,8-bis-Maleimidodiethyleneglycol, 1,11-bis-Maleimidodiethyleneglycol, 1,4 bismaleimidyl-2,3-dihydroxybutane, Dithio-bismaleimidoethane),
ii) pyridyldithiols (1,4-Di-[3′-(2′-pyridyldithio)-propionamido]butane),
iii) aryl azides (Bis-[b-(4-Azidosalicylamido)ethyl]disulfide),
iv) NHS ester/maleimides (N-(a-Maleimidoacetoxy) succinimide ester, N-[ß-Maleimidopropyloxy] succinimide ester, N-[g-Maleimidobutyryloxy]succinimide ester, N-[g-Maleimidobutyryloxy]sulfosuccinimide ester, m-Maleimidobenzoyl-N-hydroxysuccinimide ester, m-Maleimidobenzoyl-N-hydroxysulfosuccinimide ester, Succinimidyl 4[N-maleimidomethyl]cyclohexane-1-carboxylate, Sulfosuccinimidyl 4[N-maleimidomethyl]cyclohexane-1-carboxylate, [N-e-Maleimidocaproyloxy] succinimide ester, [N-e-Maleimidocaproyloxy]sulfosuccinimide ester Succinimidyl 4-[p-maleimidophenyl]butyrate, Sulfosuccinimidyl 4[p-maleimidophenyl]butyrate, Succinimidyl-6[ß-maleimidopropionamido]hexanoate, Succinimidyl-4-[N-Maleimidomethyl] cyclohexane-1-carboxy-[6-amidocaproate], N-[k-Maleimidoundecanoyloxy]sulfosuccinimide ester, succinimidyl-([N-maleimidopropionamido]-#ethyleneglycol) ester),
v) NHS ester/pyridyldithiols (4-Succinimidyloxycarbonyl-methyl-a-[2-pyridyldithio]toluene, 4-Sulfosuccinimidyl-6-methyl-a-(2-pyridyldithio)toluamidohexanoate),
vi) NHS ester/haloacetyls (N-Succinimidyl iodoacetate, Succinimidyl 3-[bromoacetamido]propionate, N-Succinimidyl[4-iodoacetyl]aminobenzoate, N-Sulfosuccinimidyl[4-iodoacetyl]aminobenzoate),
vii) pyridyldithiol/aryl azides (N-[4-(p-Azidosalicylamido) butyl]-3″-(2″-pyridyldithio)propionamide),
viii) maleimide/hydrazides (N-[ß-Maleimidopropionic acid] hydrazide, trifluoroacetic acid salt, [N-e-Maleimidocaproic acid] hydrazide, trifluoroacetic acid salt, 4-(4-N-Maleimidophenyl)butyric acid hydrazide hydrochloride, N-[k-Maleimidoundecanoic acid]hydrazide),
ix) pyridyldithiol/hydrazides (3-(2-Pyridyldithio)propionyl hydrazide),
x) isocyanate/maleimides (N-[p-Maleimidophenyl]isocyanate), and 1,6-Hexane-bis-vinylsulfone, to name but a few.

In certain embodiments of the methods, peptides, and conjugates described above, a crosslinker is a moiety derived from a bifunctional crosslinking reagent as described above. In some embodiments, a crosslinker is a moiety derived from a bifunctional crosslinking reagent capable of conjugating a surface amine of a macromolecule and a sulfhydryl of a targeting peptide. In certain embodiments, a crosslinker is a moiety derived from a bifunctional crosslinking reagent capable of conjugating a surface hydroxyl of a macromolecule and a sulfhydryl of a targeting peptide. In some embodiments, a crosslinker is a moiety derived from a bifunctional crosslinking reagent capable of conjugating a surface sulfhydryl of a macromolecule and a thiol of a targeting peptide. In some embodiments, a crosslinker is a moiety derived from a bifunctional crosslinking reagent capable of conjugating a surface carboxyl of a macromolecule and a sulfhydryl of a targeting peptide. In some embodiments, a crosslinker is a moiety having the structure:

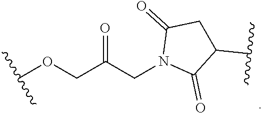 [SMILES: *OCC(=O)CN1C(=O)CC(*)C1=O]

In certain embodiments, L³ is a covalent bond. In certain embodiments, L³ is a crosslinker derived from a bifunctional crosslinking reagent capable of conjugating a reactive moiety of the (PSMAi)/chelator construct with a reactive moiety of the macromolecule. In certain embodiments, L³ is a crosslinker derived from a bifunctional crosslinking reagent capable of conjugating a reactive moiety of the bombesin/gastrin-releasing peptide receptor ligand (GRP)/chelator construct with a reactive moiety of the macromolecule. In certain embodiments, L³ is a crosslinker derived from a bifunctional crosslinking reagent capable of conjugating a sulfhydryl of the bombesin/gastrin-releasing peptide receptor ligand (GRP)/chelator construct with a reactive moiety of the macromolecule. In certain embodiments, the bifunctional crosslinking reagent is a maleimide or haloacetyl. In certain embodiments, the bifunctional crosslinking reagent is a maleimide.

In some embodiments, each -Cy-is independently an optionally substituted 5-8 membered bivalent, saturated, partially unsaturated, or aryl ring having 0-4 heteroatoms independently selected from nitrogen, oxygen, or sulfur. In some embodiments, each -Cy-is independently an optionally substituted 6-membered bivalent, saturated, partially unsaturated, or aryl ring having 0-4 heteroatoms independently selected from nitrogen, oxygen, or sulfur. In some embodiments, -Cy-is phenylene.

In certain embodiments, conjugates of the present disclosure are of the formula:

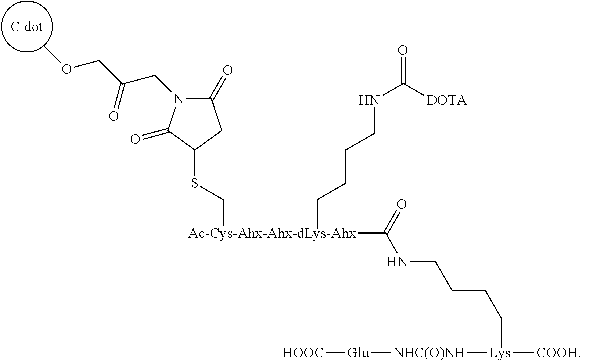

In certain embodiments, conjugates of the present disclosure are of the formula:

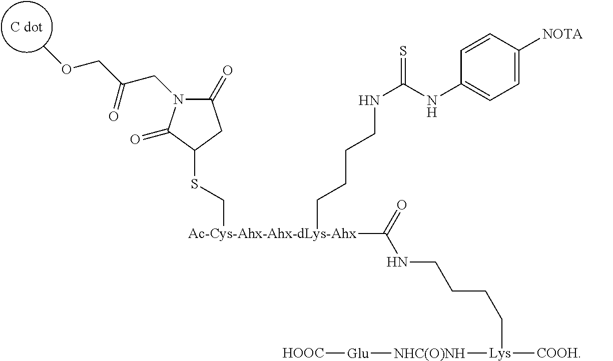

In certain embodiments, conjugates of the present disclosure are of the formula:

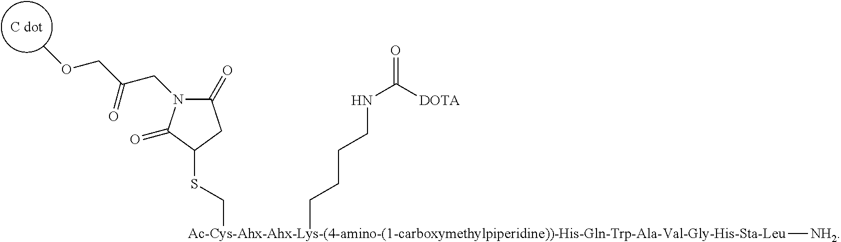

In certain embodiments, conjugates of the present disclosure are of the formula:

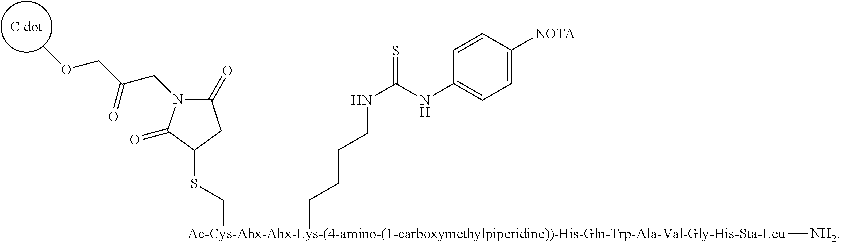

Intermediates

The present disclosure also includes intermediates useful in the synthesis of provided conjugates. Accordingly, in some embodiments the present disclosure provides a compound of formula:

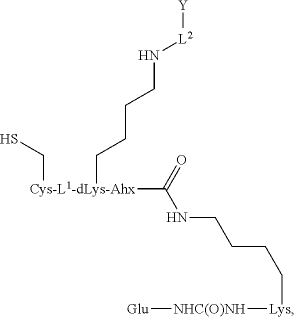

wherein each of L¹, L², and Y is as defined above and described in classes and subclasses herein, both singly and in combination.

In some embodiments, the present disclosure provides a compound of formula:

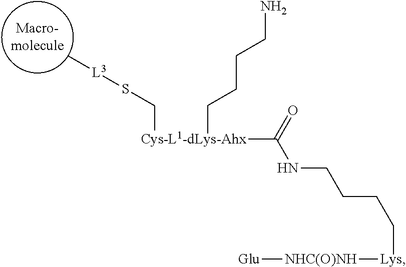

wherein each of L¹, L³, and a macromolecule is as defined above and described in classes and subclasses herein, both singly and in combination. In some embodiments, the present disclosure provides a compound of formula:

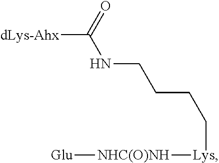

wherein one or more amino acid side chain groups or termini are optionally protected with a suitable protecting group, and wherein one amino acid is optionally attached to a resin.

In some embodiments, the present disclosure provides a compound of formula:

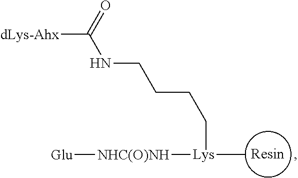

wherein one or more amino acid side chain groups or termini are optionally protected with a suitable protecting group.

In some embodiments, the present disclosure provides a compound:

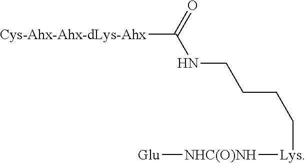

wherein one or more amino acid side chain groups or termini are optionally protected with a suitable protecting group, and wherein one amino acid is optionally attached to a resin.

In some embodiments, the present disclosure provides a compound of formula:

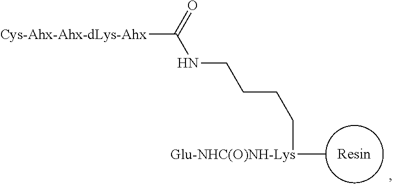

wherein one or more amino acid side chain groups or termini are optionally protected with a suitable protecting group.

In certain embodiments, the present disclosure provides a compound selected from:

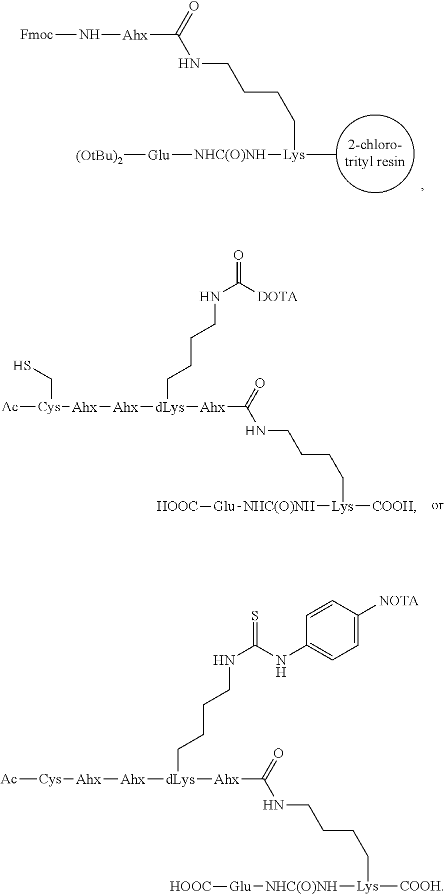

The present disclosure describes a composition (e.g., a conjugate) comprising a prostate specific membrane antigen inhibitor (PSMAi)/chelator construct covalently attached to a macromolecule (e.g., nanoparticle, e.g., polymer, e.g., protein).

In certain embodiments, the construct has the structure:

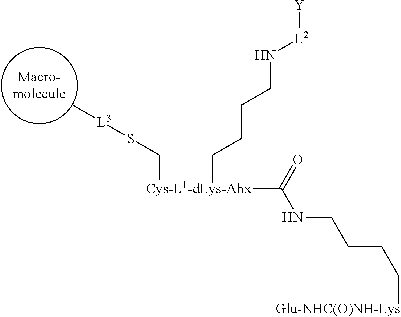

wherein: L¹ is a peptidic fragment comprising from 1 to about 10 natural or unnatural amino acid residues, or an optionally substituted, bivalent, C₁₋₂₀ saturated or unsaturated, straight or branched, hydrocarbon chain, wherein one or more methylene units of the hydrocarbon chain are optionally and independently replaced by —CHOH—, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, or —C(═NR)—; L² is an optionally substituted, bivalent, C₁₋₁₀ saturated or unsaturated, straight or branched, hydrocarbon chain, wherein one or more methylene units of the hydrocarbon chain are optionally and independently replaced by -Cy-, —CHOH—, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, or —C(═NR)—; L³ is a covalent bond or a crosslinker derived from a bifunctional crosslinking reagent capable of conjugating a reactive moiety of the (PSMAi)/chelator construct with a reactive moiety of the macromolecule, each -Cy-is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; Y is a chelator moiety; and R is hydrogen, C₁₋₆ alkyl, or a nitrogen protecting group; wherein each amino acid residue, unless otherwise indicated, may be protected or unprotected on its terminus and/or side chain group.

In certain embodiments, L¹ is a peptidic fragment comprising 1, 2, 3, 4, or 5 natural or unnatural amino acid residues. In certain embodiments, L¹ comprises one or two units of 6-aminohexanoic acid (Ahx). In certain embodiments, L¹ is -Ahx-Ahx-. In certain embodiments, L¹ is a C₁₋₁₀ saturated or unsaturated, straight or branched, hydrocarbon chain, wherein one or more methylene units of the hydrocarbon chain are optionally and independently replaced by —NR—, —O—, or —C(O)—. In certain embodiments, L¹ comprises one or more units of —(CH₂CH₂O)— or —(OCH₂CH₂)—.

In certain embodiments, L² is a C₁₋₃ saturated or unsaturated, straight or branched, hydrocarbon chain, wherein one or more methylene units of the hydrocarbon chain are optionally and independently replaced by -Cy-, —NR—, —N(R)C(O)—, —C(O)N(R)—, —O—, —C(O)—, —OC(O)—, or —C(O)O—. In certain embodiments, L² is a C₁₋₃ saturated or unsaturated, straight or branched, hydrocarbon chain, wherein one, two, or three, methylene units of the hydrocarbon chain are optionally and independently replaced by -Cy-, —NR—, or —C(O)—. In certain embodiments, -Cy-is phenylene. In certain embodiments, L² is —C(O)— or —C(O)NH-phenylene.

In certain embodiments, the chelator is DOTA. In certain embodiments, the chelator is NOTA.

In certain embodiments, L³ is derived from a bifunctional crosslinking reagent capable of conjugating a sulfhydryl on the (PSMAi)/chelator construct with a moiety of the macromolecule.

In certain embodiments, the bifunctional crosslinking reagent is a maleimide or haloacetyl. In certain embodiments, the bifunctional crosslinking reagent is a maleimide.

In certain embodiments, the macromolecule is a nanoparticle (e.g., an ultrasmall nanoparticle, e.g., a C-dot, e.g., a C′-dot). In certain embodiments, the macromolecule has a diameter no greater than 20 nm (e.g., has a diameter no greater than 15 nm, e.g., has a diameter no greater than 10 nm).

In certain embodiments, the composition comprises: a fluorescent silica-based nanoparticle comprising: a silica-based core; a fluorescent compound within the core; a silica shell surrounding a portion of the core; an organic polymer attached to the nanoparticle, thereby coating the nanoparticle, wherein the nanoparticle has a diameter no greater than 20 nm.

In certain embodiments, from 1 to 100 (e.g., from 1 to 60, e.g., from 1 to 50 e.g., from 1 to 30, e.g., from 1 to 20) PSMAi ligands are attached to the macromolecule. In certain embodiments, the the macromolecule comprises a radiolabel (e.g., ⁸⁹Zr, ⁶⁴Cu, ⁶⁸Ga, ⁸⁶Y, ¹²⁴I, ¹⁷⁷Lu, ²²⁵Ac, ²¹²Pb, ⁶⁷Cu and ²¹¹At).

In certain embodiments, the chelator comprises a member selected from the group consisting of N,N-Bis(2-hydroxy-5-(carboxyethyl)-benzyl)ethylenediamine-N,N′-diacetic acid (HBED-CC) (HBED-CC), 1,4,7,10-tetraazacyclododecane-1,4,7, 10-tetraacetic acid (DOTA), diethylenetriaminepentaacetic (DTPA), desferrioxamine (DFO), and triethylenetetramine (TETA).

In certain embodiments, the composition comprises:

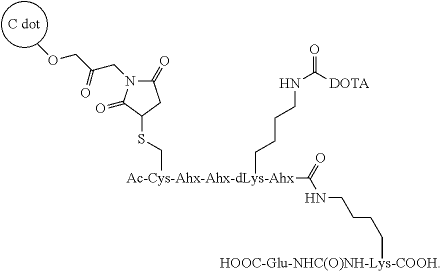

In certain embodiments, the composition comprises:

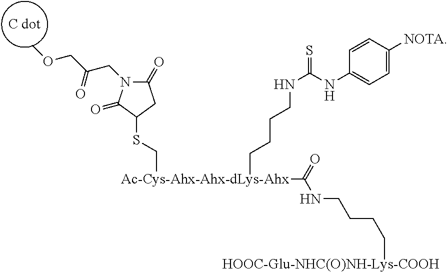

In certain embodiments, the method comprises loading orthogonally protected lysine building block comprising a suitable protecting group (e.g., Fmoc-Lys(Dde)-OH) on a resin (e.g., a 2-ClTrt resin) (e.g., in a manual reaction vessel); removing the suitable protecting group from the resin to produce a first compound; contacting (e.g., at the same time as the removing step) protected glutamic acid (e.g., di-tBU protected) with suitable reagents (e.g., triphosgene and DIEA, e.g., for 6 h at 0° C.) to produce a glutamic isocyanate building block [OCN-Glu-(OtBu)₂]; contacting (e.g., overnight, e.g., at room temperature) the isocyanate building block [OCN-Glu-(OtBu)2] with a free a amino group of the first compound to yield a fully protected urea on a second compound on the resin.

In certain embodiments, the second compound is further reacted by removing a protecting group (e.g., by 2% hydrazine) on a Lys of the second compound; obtaining a third compound by building a peptide sequence (e.g., Ac-Cys-Ahx-Ahx-dLys-Ahx-) on the ε-amino group of the Lys of the second compound; removing suitable protecting groups (e.g., with Trt for Cys and Mtt for Lys) as appropriate (e.g., via treatment with 20% Piperidine, e.g., for 10 min); optionally, assembling (e.g. And recoupling at every cycle) a peptide chain via sequential acylation (e.g., 20 min for coupling) with “in situ” activated suitably protected amino acids (e.g., where the “in situ” activated Fmoc-amino acids were carried out using with uronium salts and DIEA); removing a suitable protecting group on dLys (e.g., in the same reaction); cleaving the third compound from the resin (e.g., via treatment of TFA) to produce a fourth compound; contacting (e.g., overnight, e.g., in DMF) the fourth compound with a suitable chelator reagent (e.g., p-SCN-Bn-NOTA) in the presence of a suitable base to produce a chelator-labeled (e.g., NOTA-labeled, e.g., DOTA-labeled, e.g., HBED-CC-labeled) fifth compound; removing protecting groups from the fifth compound (e.g., via TFA, e.g., in the presence of scavengers (e.g., at a 2.5% w/v concentration) (e.g., wherein the scavengers comprise one or more of phenol, water, TIS, TA, and EDT) to produce a sixth compound (e.g., target molecule, e.g., PSMAi-NOTA, e.g., PSMAi-DOTA, e.g., PSMAi-HBED-CC); optionally purifying the sixth compound; and attaching (e.g., covalently, e.g., malemide chemistry) the sixth compound to a macromolecule (e.g., nanoparticle (e.g., C′ or C dot), e.g., polymer, e.g., protein); (e.g., selectively protecting a diprotected HBED-CC using trityl type protecting group (e.g., Trt, Cl-Trt, Mtt, Mmt) or similar).

In certain embodiments, the third compound is or comprises:

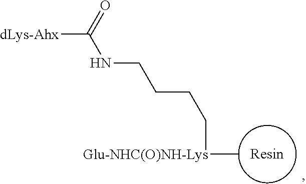

wherein one or more amino acid side chain groups or termini are optionally protected with a suitable protecting group.

In certain embodiments, the third compound is:

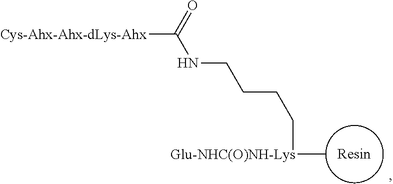

wherein one or more amino acid side chain groups or termini are optionally protected with a suitable protecting group.

The present disclosure also describes a compound:

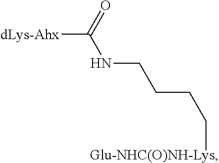

wherein one or more amino acid side chain groups or termini are optionally protected with a suitable protecting group, and wherein one amino acid is optionally attached to a resin.

The present disclosure also describes a compound:

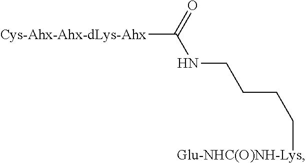

wherein one or more amino acid side chain groups or termini are optionally protected with a suitable protecting group, and wherein one amino acid is optionally attached to a resin.

The present disclosure also describes a compound selected from:

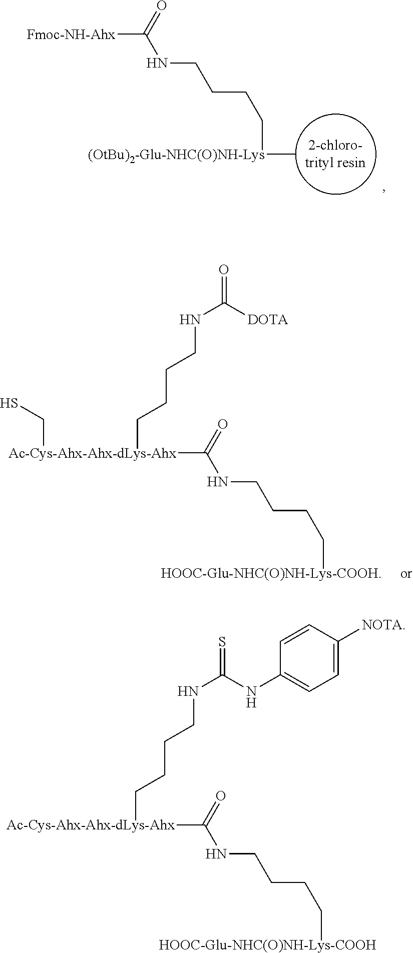

The present disclosure also describes a compound selected from:

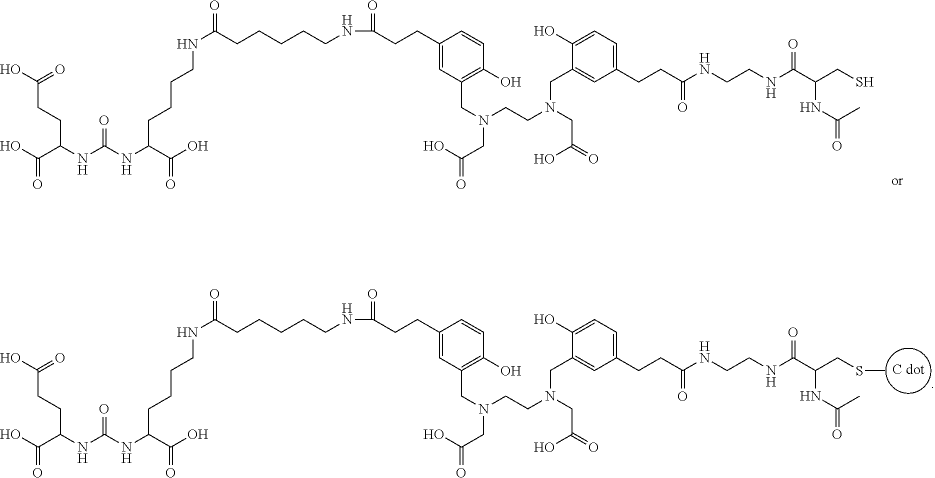 [SMILES: CC(=O)NC(CS)C(=O)NCCNC(=O)CCc1ccc(O)c(CN(CCN(CC(=O)O)Cc2cc(CCC(=O)NCCCCCC(=O)NCCCCC(NC(=O)NC(CCC(=O)O)C(=O)O)C(=O)O)ccc2O)CC(=O)O)c1 ; CSCC(NC(C)=O)C(=O)NCCNC(=O)CCc1ccc(O)c(CN(CCN(CC(=O)O)Cc2cc(CCC(=O)NCCCCCC(=O)NCCCCC(NC(=O)NC(CCC(=O)O)C(=O)O)C(=O)O)ccc2O)CC(=O)O)c1]

The present disclosure also describes a method of treating a disease or condition, the method comprising: administering to a subject a pharmaceutical composition comprising any of the compositions described herein.

In certain embodiments, the pharmaceutical composition further comprises a carrier.

The present disclosure is also directed to a method of in vivo imaging (e.g., intraoperative imaging), the method comprising: administering to a subject the composition of any one of the compositions described herein (e.g., such that the composition preferably collects in a particular region (e.g., near or within a particular tissue type, e.g., cancer tissue, e.g., prostate cancer tissue), wherein the composition comprises an imaging agent; and detecting (e.g., via PET, X-ray, MRI, CT) the imaging agent.

The present disclosure also describes a composition (e.g., a pharmaceutical composition) comprising a prostate specific membrane antigen inhibitor (PSMAi)/chelator construct covalently attached to a macromolecule (e.g., nanoparticle, e.g., polymer, e.g., protein) for use in a method of treating cancer (e.g., prostate cancer) in a subject, wherein the treating comprises delivering the composition to the subject.

The present disclosure also describes a composition (e.g., a pharmaceutical composition) comprising a prostate specific membrane antigen inhibitor (PSMAi)/chelator construct covalently attached to a macromolecule (e.g., nanoparticle, e.g., polymer, e.g., protein) for use in a method of in vivo diagnosis of cancer (e.g., prostate cancer) in a subject, the in vivo diagnosis comprises: delivering the composition to the subject (e.g., such that the composition preferably collects in a particular region (e.g., near or within a particular tissue type, e.g., cancer tissue, e.g., prostate cancer tissue), wherein the composition comprises an imaging agent; and detecting (e.g., via PET, X-ray, MRI, CT) the imaging agent.

The present disclosure also describes a composition (e.g., a pharmaceutical composition) comprising a prostate specific membrane antigen inhibitor (PSMAi)/chelator construct covalently attached to a macromolecule (e.g., nanoparticle, e.g., polymer, e.g., protein) for use in (a) a method of treating cancer in a subject or (b) a method of in vivo diagnosis of cancer in a subject, wherein the method comprises: delivering the composition to the subject (e.g., such that the composition preferably collects in a particular region (e.g., near or within a particular tissue type, e.g., cancer tissue, e.g., prostate cancer tissue), wherein the composition comprises an imaging agent; and detecting (e.g., via PET, X-ray, MRI, CT) the imaging agent.

The present disclosure also describes a composition (e.g., a pharmaceutical composition) comprising a prostate specific membrane antigen inhibitor (PSMAi)/chelator construct covalently attached to a macromolecule (e.g., nanoparticle, e.g., polymer, e.g., protein) for use in therapy.

The present disclosure also describes a composition (e.g., a pharmaceutical composition) comprising a prostate specific membrane antigen inhibitor (PSMAi)/chelator construct covalently attached to a macromolecule (e.g., nanoparticle, e.g., polymer, e.g., protein) for use in in vivo diagnosis.

In certain embodiments, the macromolecule is a nanoparticle (e.g., an ultrasmall nanoparticle, e.g., a C-dot, e.g., a C′-dot). In certain embodiments, the macromolecule has a diameter no greater than 20 nm (e.g., has a diameter no greater than 15 nm, e.g., has a diameter no greater than 10 nm). In certain embodiments, the macromolecule comprises: a fluorescent silica-based nanoparticle comprising: a silica-based core; a fluorescent compound within the core; a silica shell surrounding a portion of the core; an organic polymer attached to the nanoparticle, thereby coating the nanoparticle, wherein the nanoparticle has a diameter no greater than 20 nm.

In certain embodiments, from 1 to 20 PSMAi ligands are attached to the macromolecule.

In certain embodiments, the composition comprises a radiolabel (e.g., ⁸⁹Zr, ⁶⁴Cu, ⁶⁸Ga, ⁸⁶Y, ¹²⁴l, ¹⁷⁷Lu, ²²⁵Ac, ²¹²Pb, and ²¹¹At).

In certain embodiments, the chelator comprises a member selected from the group consisting of N,N′-Di(2-hydroxybenzyl)ethylenediamine-N,N′-diacetic acid monohydrochloride (HBED-CC), 1,4,7,10-tetraazacyclododecane-1,4,7, 1 0-tetraacetic acid (DOTA), diethylenetriaminepentaacetic (DTPA), desferrioxamine (DFO), and triethylenetetramine (TETA).

In certain embodiments, the composition comprises:

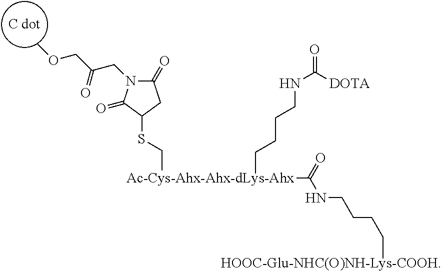

In certain embodiments, the composition comprises:

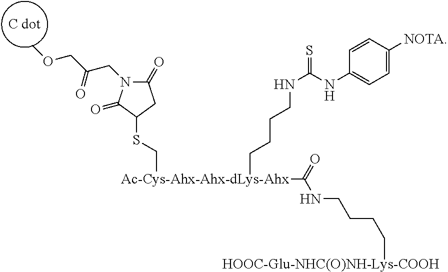

The present disclosure also describes a composition (e.g., a conjugate) comprising a bombesin/gastrin-releasing peptide receptor ligand (GRP)/chelator construct covalently attached to a macromolecule (e.g., nanoparticle, e.g., polymer, e.g., protein).

In certain embodiments, the bombesin/gastrin-releasing peptide receptor ligand (GRP)/chelator construct comprises a peptide of the sequence:

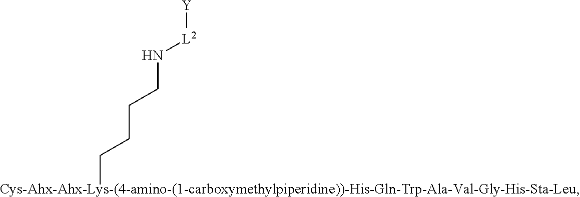

wherein: L² is an optionally substituted, bivalent, C₁₋₁₀ saturated or unsaturated, straight or branched, hydrocarbon chain, wherein one or more methylene units of the hydrocarbon chain are optionally and independently replaced by -Cy-, —CHOH—, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, or —C(═NR)—; each -Cy-is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; Y is a chelator moiety; and R is hydrogen, C₁₋₆ alkyl, or a nitrogen protecting group; wherein each amino acid residue, unless otherwise indicated, may be protected or unprotected on its terminus and/or side chain group.

In certain embodiments, the bombesin/gastrin-releasing peptide receptor ligand (GRP)/chelator construct is covalently attached via the depicted cysteine residue to the macromolecule through L³, wherein, L³ is a covalent bond or a crosslinker derived from a bifunctional crosslinking reagent capable of conjugating a reactive moiety of the bombesin/gastrin-releasing peptide receptor ligand (GRP)/chelator construct with a reactive moiety of the macromolecule.

In certain embodiments, the bombesin/gastrin-releasing peptide receptor ligand (GRP)/chelator construct has the structure:

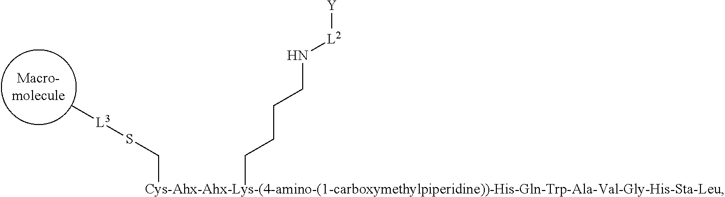

wherein each of L², L³, Y is as defined above and described in classes and subclasses herein, both singly and in combination.

In certain embodiments, L³ is derived from a bifunctional crosslinking reagent capable of conjugating a sulfhydryl on the bombesin/gastrin-releasing peptide receptor ligand (GRP)/chelator construct with a moiety of the macromolecule. In certain embodiments, the bifunctional crosslinking reagent is a maleimide or haloacetyl. In certain embodiments, the bifunctional crosslinking reagent is a maleimide.

In certain embodiments, L₂ is a covalent bond.

In certain embodiments, the chelator is DOTA. In certain embodiments, the chelator is NOTA.

In certain embodiments, the macromolecule is a nanoparticle (e.g., an ultrasmall nanoparticle, e.g., a C-dot, e.g., a C′-dot). In certain embodiments, the macromolecule has a diameter no greater than 20 nm (e.g., has a diameter no greater than 15 nm, e.g., has a diameter no greater than 10 nm). In certain embodiments, the macromolecule comprises: a fluorescent silica-based nanoparticle comprising: a silica-based core; a fluorescent compound within the core; a silica shell surrounding a portion of the core; an organic polymer attached to the nanoparticle, thereby coating the nanoparticle, wherein the nanoparticle has a diameter no greater than 20 nm.

In certain embodiments, from 1 to 100 (e.g., from 1 to 60, e.g., from 1 to 50 e.g., from 1 to 30, e.g., from 1 to 20) bombesin/gastrin-releasing peptide receptor ligand are attached to the macromolecule.

In certain embodiments, the composition further comprises a radiolabel (e.g., ⁸⁹Zr, ⁶⁴Cu, ⁶⁸Ga, ⁸⁶Y, 124I, ¹⁷⁷Lu, ²²⁵Ac, ²¹²Pb, ⁶⁷Cu and ²¹¹At).

In certain embodiments, the chelator comprises a member selected from the group consisting of N,N′-Bis(2-hydroxy-S-(carboxyethyl)-benzyl)ethylenediamine-N,N′-di acetic acid (HBED-CC) (HBED-CC), 1,4,7,10-tetraazacyclododecane-1,4,7, 10-tetraacetic acid (DOTA), diethylenetriaminepentaacetic (DTPA), desferrioxamine (DFO), and triethylenetetramine (TETA).

In certain embodiments, the composition comprises:

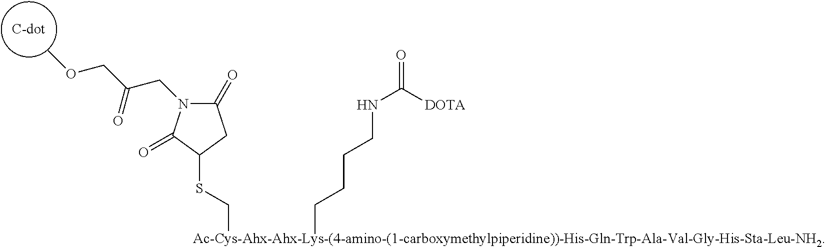

In certain embodiments, the composition comprises:

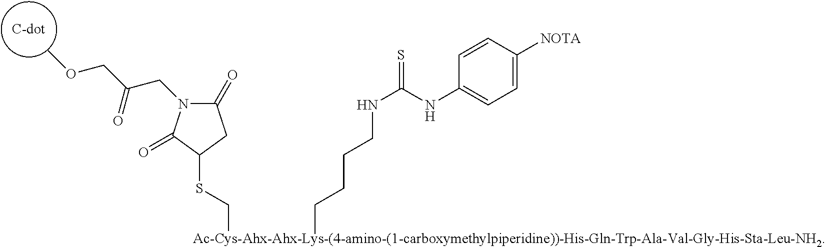

The present disclosure also describes a method of treating a disease or condition, the method comprising: administering to a subject a pharmaceutical composition comprising a composition described herein (e.g., to target a particular type of tissue (e.g., cancer tissue) (e.g., prostate cancer tissue). In certain embodiments, the pharmaceutical composition further comprises a carrier.

### Experimental Example

### Combination Therapy for Treatment of Prostate Cancer

The present example describes a combination therapy for prostate cancer using hormone therapy and ferroptotic induction.

Subjects having castration-resistant prostate cancer can develop resistance to hormone therapy, and exhibit increased expression of PSMA. Interestingly, it was found that a combinational treatment comprising hormone therapy and ferroptotic induction by nanoparticles increased cell death of cancerous cells. In particular, it was found that delaying administration of the nanoparticles about five days after hormone therapy resulted in enhanced ferroptotic induction. Hormone therapy was administered to the subject daily.

Although the Example uses nanoparticles to induce ferroptosis, other agents that induce ferroptosis can be used. Moreover, although this Example uses enzalutamide as hormone therapy, other types of hormone therapies can be used. This type of combination therapy can also be effective for other types of diseases and/or cancers.

The present Example shows data where LNCAP tumors were pre-treated with enzalutamide, an antiandrogen that targets androgens like testosterone and dihydrotestosterone, until optimum PSMA expression was achieved, and prior to administering PSMAi-C′ dots. Without wishing to be bound to any theory, the data suggests that increased PSMA expression enhances particle uptake and therefore cell death to a greater degree than without cell “priming” by enzalutamide.

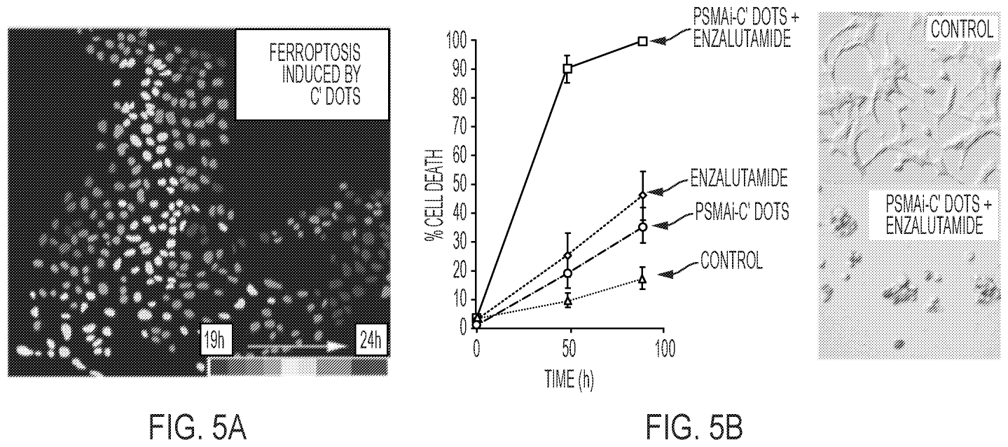

FIGS. 5A-5B show that C′ dot nanoparticles induce ferroptosis that spreads through cell populations and kills prostate cancer cells in combination with Enzalutamide.

FIG. 5A shows that C′ dot-treated cells undergo ferroptotic cell death that spreads through entire populations in a wave-like manner. The image shows nuclei of dead cells, pseudocolored to indicate the timing of cell death after treatment (from 19-24 hours).

FIG. 5B shows that prostate cancer-targeted PSMAi-C′ dots kill androgen-dependent prostate cancer cells (LNCaP) efficiently when combined with enzalutamide. Images show representative control and PSMAi-C′ dot+enzalutamide-treated LNCaP cells.

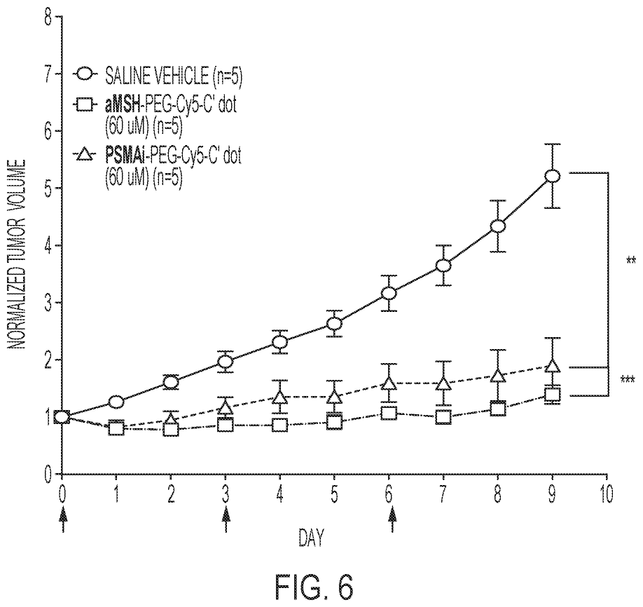

FIG. 6 shows that C′ dot ferroptotic induction inhibits in vivo prostate cancer growth. Male, LNCAP tumor-bearing mice, were intravenously (IV) administered saline vehicle, 60 μM PSMAi-PEG-Cy5-C′ dots, 60 μM αMSH-PEG-Cy5-C′ dots on days 0, 3, and 6 (200 μl/injection). Tumor growth was monitored over time use caliper measurements, and volumes were calculated using: Volume=(Long Axis×Short Axis²)/2. Total tumor volumes were normalized to Day 0 volumes and plotted using GraphPad Prism software. The results demonstrate that the administration of functionalized (PSMAi, αMSH) C′ dots results in a significant inhibition of LNCAP tumor growth when compared to vehicle control. ** p=0.0021; ***p=0.0002.

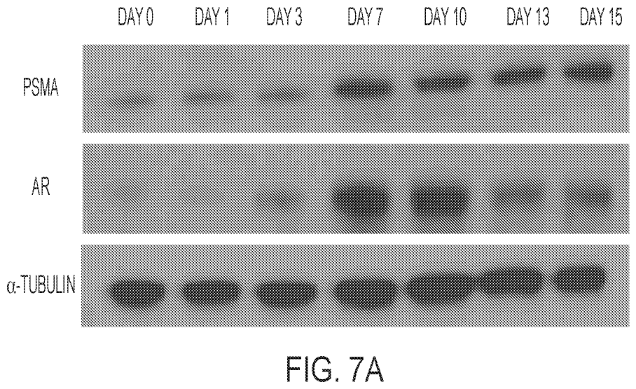

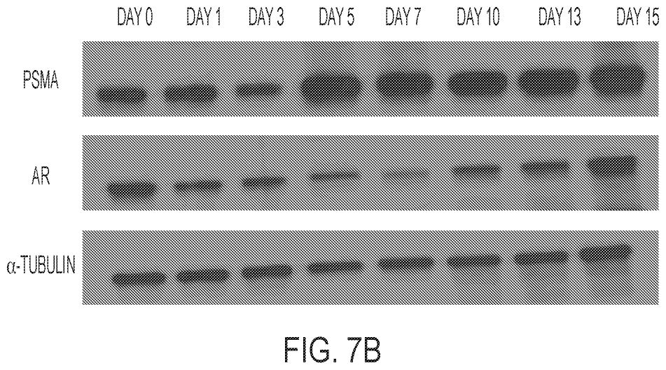

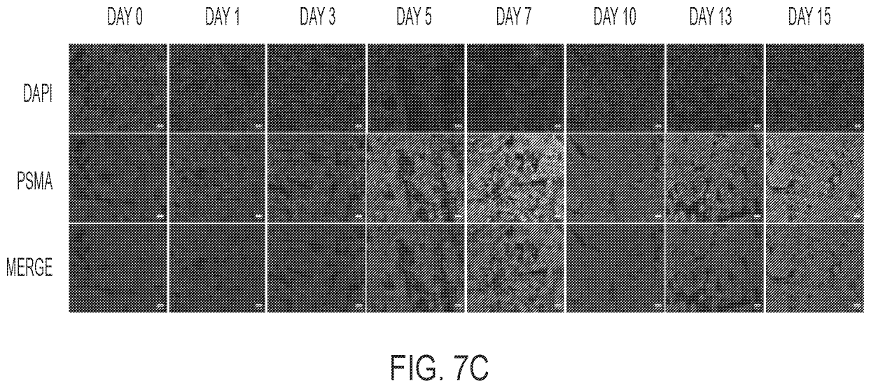

FIGS. 7A-7C show that enzalutamide exposure increases PSMA expression in vitro and in vivo.

FIG. 7A shows a Western Blot of LNCAP prostate cancer cells that were continuously exposed to 10 μM enzalutamide in vitro for 15 days. At the conclusion of exposure, cells were collected and the expression levels of PSMA and AR were examined via Western blot. The results demonstrate an increase in PSMA expression occurs on, or around, Day 7 of exposure. A transient increase in AR expression is also observed from Day 7 to Day 10.

FIG. 7B shows that, similar to results demonstrated in FIG. 7A, daily administration of enzalutamide (10 mg/kg/day; oral gavage) to mice bearing LNCAP xenografts also resulted in an increase in PSMA expression at Day 5.

FIG. 7C shows images of tumor sections that were collected from mice that were also used to evaluate PSMA expression using immunofluorescence staining. Staining for PSMA in LNCAP xenografts again supports an increase in PSMA expression at Day 5, demonstrated as an increase in fluorescence signal. Together, these data illustrate that exposure to enzalutamide results in an increase in the expression of PSMA both in vitro and in vivo and indicate that an optimal window for the co-administration of functionalized C′ dots with enzalutamide for combination therapy.

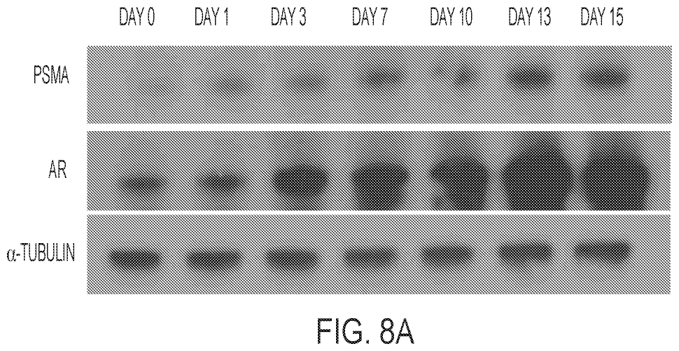

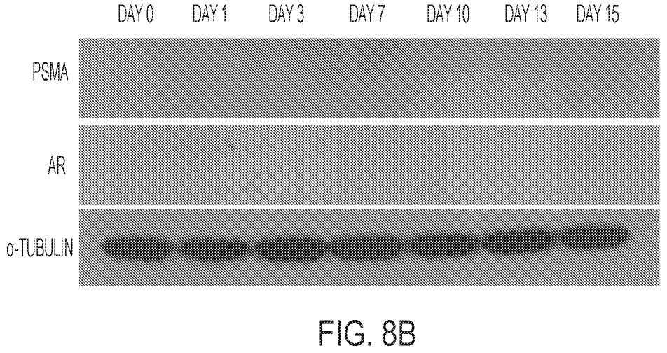

FIGS. 8A-8B show Western Blots indicating that exposure to enzalutamide increases PSMA expression in LNCAP-AR (PSMA+) but not PC-3 (PSMA−) control cell lines in vitro.

FIG. 8A shows a Western Blot of LNCAP-AR prostate cancer cells that were utilized as an anti-androgen (enzalutamide) resistant control line. Continuous exposure to 10 μM enzalutamide in vitro over the course of 15 days resulted in an time-dependent increase in PSMA expression, a result similar to observations in parental LNCAP cells. Interestingly, a clear increase in AR expression is observed from Day 3 onward, most likely due to androgen depravation resulting from enzalutamide exposure.

FIG. 8B shows a Western Blot of PC-3 prostate cancer cells, which are negative for PSMA expression, that were utilized as a second control cell line. PC-3 cells exposed to 10 μM enzalutamide for 15 days in vitro demonstrate a lack of PSMA expression at all time points. Additionally, PC-3 cells are also negative for AR across all tested time points. Taken together, these results robustly support that exposure to enzalutamide results in an increase in PSMA expression, in PSMA+ cell lines, irrespective of AR expression levels.

To summarize, without wishing to be bound to any theory, this data shows that Enzalutamide treatment of PSMA-expressing cells may better inform in vivo treatment of prostate cancer.

1. A method of combination treatment of a subject, the method comprising:
a first step of administering an initial dose of a first agent to the subject; and
a second step of administering an initial dose of a second agent to the subject to induce ferroptosis of cancer cells,
wherein the step of administering the initial dose of the second agent occurs at a discrete period of time after the step of administering the initial dose of the second agent.

2. The method of claim 1, wherein the first agent comprises an androgen inhibitor or other agent administered as part of hormone therapy.

3-6. (canceled)

7. The method of claim 1, wherein administering the second agent induces ferroptotic rupture of the cancer cells.

8. (canceled)

9. The method of claim 1, comprising
maintaining the cancer cells in a nutrient-deprived environment, or, alternatively, not maintaining the cancer cells in a nutrient-deprived environment.

10-15. (canceled)

16. The method of claim 1, wherein the second agent comprises a nanoparticle.

17. The method of claim 16, wherein the nanoparticle has from 1 to 100 targeting ligands attached thereto.

18. The method of claim 17, wherein the targeting ligands comprise a member selected from the group comprising of PSMAi and alpha-MSH.

19. (canceled)

20. The method of claim 16, wherein the nanoparticle has an average diameter no greater than about 50 nm.

21-29. (canceled)

30. The method of claim 1, wherein the method comprises administering the second agent to the subject for accumulation at sufficiently high concentration in cancer cells to induce ferroptosis.

31. The method of claim 1, wherein the cancer cells are selected from the group consisting of renal, prostate, melanoma, pancreatic, lung, fibrosarcoma, breast, brain, ovarian, and colon cancer cells.

32-38. (canceled)

39. A method of combination treatment of a subject, the method comprising:
a first step of administering an initial dose of a first agent to the subject; and
a second step of administering an initial dose of a second agent to the subject to induce ferroptosis of cancer cells,
wherein the step of administering the initial dose of the second agent occurs at a discrete period of time after the step of administering the initial dose of the second agent,
wherein the subject has also received an androgen inhibitor, e.g., via one or more separate doses.

40. The method of claim 39, wherein the first agent comprises an androgen inhibitor or other agent administered as part of hormone therapy.

41-42. (canceled)

43. The method of claim 39, wherein the second agent comprises a ferroptosis-inducing nanoparticle with a PSMA-targeting ligand.

44. The method of claim 43, wherein an increased expression of PSMA by prostate cancer cells results in enhanced ferroptotic induction by the second agent.

45. (canceled)

46. A kit comprising:
a first agent in a unit dosage effective to treat prostate cancer in a subject receiving therapy with the first agent; and
a second agent.

47. The kit of claim 46, wherein the first agent comprises an androgen inhibitor or other agent administered as part of hormone therapy.

48. (canceled)

49. The kit of claim 46, wherein the second agent comprises a ferroptosis-inducing nanoparticle with a PSMA-targeting ligand.

50. (canceled)

51. A method of treating cancer in a subject, the method comprising:
administering a composition to the subject to induce ferroptosis of cancer cells.

52. The method of claim 51, wherein the composition comprises a nanoparticle.

53-56. (canceled)
